(19) United States
(12) Patent Application Publication
(10) Pub. No.: US 2025/0388914 A1
(43) Pub. Date: Dec. 25, 2025

(54) UV-RESISTANT BIOLOGICAL DEVICES AND EXTRACTS AND METHODS FOR PRODUCING AND USING THE SAME

(71) Applicant: Bio Capital Holdings, LLC, Houston, TX (US)

(72) Inventor: Raul CUERO RENGIFO, Cypress, TX (US)

(21) Appl. No.: 19/312,528

(22) Filed: Aug. 28, 2025

Related U.S. Application Data
(63) Continuation of application No. 18/187,249, filed on Mar. 21, 2023, now Pat. No. 12,404,511.
Application No. 18/187,249 is a continuation of application No. 16/686,326, filed on Nov. 18, 2019, now Pat. No. 11,639,505.
Application No. 16/686,326 is a continuation-in-part of application No. PCT/US2018/033090, filed on May 17, 2018.
(60) Provisional application No. 62/507,946, filed on May 18, 2017.
(60) Provisional application No. 62/557,217, filed on Sep. 12, 2017.

(51) Int. Cl. C12N 15/64; C12N 9/04; C12N 9/12

(52) U.S. Cl. CPC C12N 15/64; C12N 9/0006; C12N 9/1205; C12N 2800/10

(57) ABSTRACT

Described herein are UV-resistant or UV-protective biological devices and extracts produced therefrom. The biological devices include microbial cells transformed with a DNA construct containing genes for producing UV-resistant proteins such as, for example, hexokinase, heat shock proteins, alcohol dehydrogenase, transferrin, flavonol synthase, zinc oxidase, and iron oxidase. Methods for producing and using the devices are also described herein. Finally, compositions and methods for using the devices and extracts to reduce or prevent UV-induced damage or exposure to materials, items, plants, and human and animal subjects are described herein.

### CROSS REFERENCE TO RELATED APPLICATIONS

This application is a continuation application of U.S. application Ser. No. 18/187,249 filed on Mar. 21, 2023, which is a continuation application of U.S. application Ser. No. 16/686,326 filed on Nov. 18, 2019, which is a continuation-in-part of international application no. PCT/US2018/033090 filed on May 17, 2018, which claims priority upon U.S. provisional application Ser. Nos. 62/507,946 filed on May 18, 2017 and 62/557,217 filed Sep. 12, 2017. These applications are hereby incorporated by reference in their entirety.

### CROSS REFERENCE TO SEQUENCE LISTING

This application contains a sequence listing filed in ST.26 format entitled “930201-1041_Sequence_Listing.xml” created on May 16, 2023 and having a size of 72,246 bytes. The content of the sequence listing is incorporated herein in its entirety.

### BACKGROUND

Exposure to UV radiation causes harmful effects in a wide variety of things, both living and non-living. For example, exposure of human skin to UV radiation can cause severe sunburn and skin cancer and exposure of beneficial microorganisms to UV radiation can kill them. UV radiation can also cause materials to degrade prematurely and thus suffer mechanical failure or otherwise become unable to serve their intended purpose.

The harmful effects of UV radiation can generally be prevented or lessened through the simple step of using a compound or composition to absorb all or a portion of the UV radiation before it reaches the item it may harm. For example, chemicals in sunscreen absorb a portion of the UV radiation that would normally reach the skin and, as a result, help protect the skin from sunburn and skin cancer.

Although numerous substances capable of absorbing UV radiation are known, not all of them are suitable for all possible uses. Further, some substances may be expensive to produce or may have harmful side effects, such as toxicity or undesired chemical reactions with a protected material. Other substances simply do not last long enough in the environment in which they are used, or persist long after their period of usefulness.

Accordingly, there is a demand for new substances able to absorb UV radiation, particularly if those substances are biocompatible. The present invention addresses this demand.

### SUMMARY

Described herein are UV-resistant or UV-protective biological devices and extracts produced therefrom. The biological devices include microbial cells transformed with a DNA construct containing genes for producing UV-resistant proteins such as, for example, hexokinase, heat shock proteins, alcohol dehydrogenase, transferrin, flavonol synthase, zinc oxidase, and iron oxidase.

Methods for producing and using the devices are also described herein. Finally, compositions and methods for using the devices and extracts to reduce or prevent UV-induced damage or exposure to materials, items, plants, and human and animal subjects are described herein.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIGS. 1A and 1B shows, respectively, a linear and circular schematic of a constructed pYES2 plasmid showing the direction, placement, and size of genetic parts used in one aspect of an exemplary DNA device described herein.

FIGS. 2A and 2B shows, respectively, a linear and circular schematic of a constructed pYES2 plasmid showing the direction, placement, and size of genetic parts used of an exemplary DNA device described herein.

FIGS. 3A and 3B shows, respectively, a linear and circular schematic of a constructed pYES2 plasmid showing the direction, placement, and size of genetic parts used of an exemplary DNA device described herein.

### DETAILED DESCRIPTION

Before the present compounds, compositions, articles, devices, and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting. In this specification and in the claims that follow, reference will be made to a number of terms that shall be defined to have the following meanings: It must be noted that, as used in the specification and the appended claims, the singular forms “a,” “an,” and “the” include plural referents unless the context clearly dictates otherwise. Thus, for example, reference to “an isolated nucleic acid” includes mixtures of two or more such nucleic acids, and the like.

“Optional” or “optionally” means that the subsequently described event or circumstance can or cannot occur, and that the description includes instances where the event or circumstance occurs and instances where it does not. For example, the phrase “optionally includes a gene for a selective marker” means that the gene may or may not be present.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the endpoints of each of the ranges are significant both in relation to the other endpoint, and independently of the other endpoint.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. For example, if a bacterium is disclosed and discussed and a number of different compatible bacterial plasmids are discussed, each and every combination and permutation of bacterium and bacterial plasmid that is possible is specifically contemplated unless specifically indicated to the contrary. For example, if a class of molecules A, B, and C are disclosed as well as a class of molecules D, E, and F, and an example of a combination molecule, A-D, is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E is specifically contemplated and should be considered disclosed from disclosure of A, B, and C; D, E, and F; and the example combination

A-D. This concept applies to all aspects of this disclosure including, but not limited to, steps in methods of making and using the disclosed compositions. Thus, if a variety of additional steps can be performed, it is understood that each of these additional steps can be performed with any specific embodiment or combination of embodiments of the disclosed methods, and that each such combination is specifically contemplated and should be considered disclosed.

References in the specification and concluding claims to parts by weight of a particular element or component in a composition or article, denote the weight relationship between the element or component and any other elements or components in the composition or article for which a part by weight is expressed.

Thus, in a compound containing 2 parts by weight of component X and 5 parts by weight of component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

“Heterologous” genes and proteins are genes and proteins that have been experimentally put into a cell that are not normally expressed by that cell. A heterologous gene may be cloned or derived from a different cell type or species than the recipient cell or organism. Heterologous genes may be introduced into cells by transduction or transformation.

An “isolated” nucleic acid is one that has been separated from other nucleic acid molecules and/or cellular material (peptides, proteins, lipids, saccharides, and the like) normally present in the natural source of the nucleic acid. An “isolated” nucleic acid may optionally be free of the flanking sequences found on either side of the nucleic acid as it naturally occurs. An isolated nucleic acid can be naturally occurring, can be chemically synthesized, or can be a cDNA molecule (i.e., is synthesized from an mRNA template using reverse transcriptase and DNA polymerase enzymes.

“Transformation” or “transfection” as used herein refers to a process for introducing heterologous DNA into a host cell. Transformation can occur under natural conditions or may be induced using various methods known in the art. Many methods for transformation are known in the art and the skilled practitioner will know how to choose the best transformation method based on the types of cells being transformed. Methods for transformation include, for example, viral infection, electroporation, lipofection, chemical transformation, and particle bombardment. Cells may be stably transformed (i.e., the heterologous DNA is capable of replicating as an autonomous plasmid or as part of the host chromosome) or may be transiently transformed (i.e., the heterologous DNA is expressed only for a limited period of time).

“Competent cells” refers to microbial cells capable of taking up heterologous DNA. Competent cells can be purchased from a commercial source, or cells may be made competent using procedures known in the art. Exemplary procedures for producing competent cells are provided in the Examples.

### DNA Constructs and Biological Devices

The biological devices described herein can be used to produce UV-protective proteins, extracts, and other components. The devices are generally composed of host cells, where the host cells are transformed with a DNA construct described herein that promotes the expression of proteins involved in UV resistance responses.

It is understood that one way to define the variants and derivatives of the genetic components and DNA constructs described herein is through defining the variants and derivatives in terms of homology/identity to specific known sequences. Those of skill in the art readily understand how to determine the homology of two nucleic acids. For example, the homology can be calculated after aligning two sequences so that the homology is at its highest level. Another way of calculating homology can be performed by published algorithms (see Zuker, M., 1989 Science, 244:48-52; Jaeger et al., 1989, Proc. Natl. Acad. Sci. USA, 86:7706-7710; Jaeger et al., 1989, Methods Enzymol., 183:281-306, which are herein incorporated by reference for at least material related to nucleic acid alignment.)

As used herein, “conservative” mutations are mutations that result in an amino acid change in the protein produced from a sequence of DNA. When a conservative mutation occurs, the new amino acid has similar properties as the wild type amino acid and generally does not drastically change the function or folding of the protein (e.g., switching isoleucine for valine is a conservative mutation since both are small, branched, hydrophobic amino acids). “Silent mutations,” meanwhile, change the nucleic acid sequence of a gene encoding a protein but do not change the amino acid sequence of the protein.

It is understood that the description of conservative mutations an homology can be combine together in any combination, such as embodiments that have at least 70%, 75%, 80%, 85%, 90%, 95%, or 99% homology to a particular sequence wherein the variants are conservative mutations. It is understood that any of the sequences described herein can be a variant or derivative having the homology values listed above. In one aspect, the separate elements of the DNA constructs disclosed herein have at least 90% homology with the sequences disclosed herein. In another aspect, the separate elements have at least 95% homology or at least 99% homology with the sequences disclosed herein.

In one aspect, a database such as, for example, GenBank, can be used to determine the sequences of genes and/or regulatory regions of interest, the species from which these elements originate, and relate homologous sequences.

In one aspect, the DNA construct comprises the following genetic components: (a) a gene that expresses hexokinase, (b) a gene that expresses a heat shock protein, (c) a gene that expresses alcohol dehydrogenase, and (d) a gene that expresses transferrin.

In another aspect, the DNA construct comprises the following genetic components: (a) a gene that expresses zinc oxidase or a gene that expresses flavonol synthase, (b) a gene that expresses hexokinase, (c) a gene that expresses a heat shock protein, (d) a gene that expresses alcohol dehydrogenase, and (e) a gene that expresses iron oxidase.

In one aspect, the DNA construct described herein can promote the expression of UV-resistant proteins. In one aspect, the DNA construct is, from 5′ to 3′, the following genetic components in the following order: (a) a gene that expresses hexokinase, (b) a gene that expresses a heat shock protein, (c) a gene that expresses alcohol dehydrogenase, and (d) a gene that expresses transferrin.

In still another aspect, the DNA construct is, from 5′ to 3′, the following genetic components in the following order: (a) a gene that expresses flavonol synthase, (b) a gene that expresses hexokinase, (c) a gene that expresses a heat shock protein, (d) a gene that expresses alcohol dehydrogenase, and (e) a gene that expresses iron oxidase.

In still another aspect, the DNA construct is, from 5′ to 3′, the following genetic components in the following order: (a) a gene that expresses zinc oxidase, (b) a gene that expresses hexokinase, (c) a gene that expresses a heat shock protein, (d) a gene that expresses alcohol dehydrogenase, and (e) a gene that expresses iron oxidase.

In one aspect, a regulatory sequence is already incorporated into a vector such as, for example, a plasmid, prior to genetic manipulation of the vector. In another aspect, the regulatory sequence can be incorporated into the vector through the use of restriction enzymes or any other technique known in the art. In one aspect, the regulatory sequence is a promoter. The term “promoter” refers to a DNA sequence capable of controlling the expression of a coding sequence. In one aspect, the coding sequence to be controlled is located 3′ to the promoter. In another aspect, the promoter is derived from a native gene. In an alternative aspect, the promoter is composed of multiple elements derived from different genes and/or promoters. A promoter can be assembled from elements found in nature, from artificial and/or synthetic elements, or from a combination thereof. It is understood by those skilled in the art that different promoters can direct the expression of a gene in different tissues or cell types, at different stages of development, in response to different environmental or physiological conditions, and/or in different species. In one aspect, the promoter functions as a switch to activate the expression of a gene.

In one aspect, the promoter is “constitutive.” A constitutive promoter is a promoter that causes a gene to be expressed in most cell types at most times. In another aspect, the promoter is “regulated.” A regulated promoter is a promoter that becomes active in response to a specific stimulus. A promoter may be regulated chemically, for example, in response to the presence of a particular metabolite (e.g., lactose or tryptophan), a metal ion, a molecule secreted by a pathogen, or the like. A promoter may also be regulated physically, for example, in response to heat, cold, water stress, salt stress, oxygen concentration, illumination (including UV exposure), wounding, or the like.

Promoters that are useful to drive expression of the nucleotide sequences described herein are numerous and familiar to those skilled in the art. Suitable promoters include, but are not limited to, the following: T3 promoter, T7 promoter, iron promoter, and GAL1 promoter. Variants of these promoters are also contemplated. In one aspect, the promoter is a GAL1 promoter. In another aspect, several promoters, either the same or different, can appear in the same device. The skilled artisan will be able to use site-directed mutagenesis and/or other mutagenesis techniques to modify the promoters to promote more efficient function. The promoter can be positioned, for example, from 10-100 nucleotides away from a ribosomal binding site. In one aspect, the promoter is positioned before the gene that expresses hexokinase, heat shock protein, alcohol dehydrogenase, transferrin, flavonol synthase, iron oxidase, zinc oxidase, or any combination thereof.

In one aspect, the promoter is a GAL1 promoter. In another aspect, the GAL1 promoter is native to the plasmid used to create the vector. In another aspect, a GAL1 promoter is positioned before the gene that expresses hexokinase, heat shock protein, alcohol dehydrogenase, flavonol synthase, and zinc oxidase. In another aspect, the promoter is a GAL1 promoter obtained from or native to the pYES2 plasmid. In another aspect, an iron promoter is positioned before the gene that expresses transferrin and iron oxidase.

In another aspect, the regulatory sequence is a terminator or stop sequence. As used herein, a terminator is a sequence of DNA that marks the end of a gene or operon to be transcribed. In a further aspect, the terminator is an intrinsic terminator or a Rho-dependent transcription terminator. As used herein, an “intrinsic terminator” is a sequence wherein a hairpin structure can form in the nascent transcript and wherein the hairpin disrupts the mRNA/DNA/RNA polymerase complex. As used herein, a “Rho-dependent” transcription terminator requires a Rho factor protein complex to disrupt the mRNA/DNA/RNA polymerase complex. In one aspect, the terminator is a CYC1 terminator. In still another aspect, multiple terminators can be included in the same DNA construct.

In a further aspect, the regulatory sequence includes both a promoter and a terminator or stop sequence. In a still further aspect, the regulatory sequence can include multiple promoters or terminators. Other regulatory elements, such as enhancers, are also contemplated. Enhancers may be located from about 1 to about 2,000 nucleotides in the 5′ direction from the start codon of the DNA to be transcribed, or may be located 3′ to the DNA to be transcribed. Enhancers may be “cis-acting,” that is, located on the same molecule of DNA as the gene whose expression they affect.

In certain aspects, the DNA construct can include a gene that expresses a reporter protein. The selection of the reporter protein can vary. For example, the reporter protein can be a yellow fluorescent protein, a red fluorescent protein, a green fluorescent protein, or a cyan fluorescent protein. The amount of fluorescence that is produced by the biological device can be correlated to the amount of DNA incorporated into the host cells. The fluorescence produced by the device can be detected and quantified using techniques known in the art. For example, spectrofluorometers are typically used to measure fluorescence.

In one aspect, the DNA construct is, from 5′ to 3′, the following genetic components in the following order: (1) a gene that expresses hexokinase having SEQ

ID NO. 4 or at least 70% homology thereto, (2) a gene that expresses a heat shock protein having SEQ ID NO. 5 or at least 70% homology thereto, (3) a gene that expresses alcohol dehydrogenase having SEQ ID NO. 6 or at least 70% homology thereto, (4) an iron promoter having SEQ ID NO. 7 or at least 70% homology thereto, and (5) a gene that expresses transferrin having SEQ ID NO. 8 or at least 70% homology thereto.

In another aspect, the DNA construct is, from 5′ to 3′, the following genetic components in the following order: (1) a gene that expresses flavonol synthase having SEQ ID NO. 9 or at least 70% homology thereto, (2) a gene that expresses hexokinase having SEQ ID NO. 4 or at least 70% homology thereto, (3) a gene that expresses a heat shock protein having SEQ ID NO. 5 or at least 70% homology thereto, (4) a gene that expresses alcohol dehydrogenase having SEQ ID NO. 6 or at least 70% homology thereto, (5) an iron promoter having SEQ ID NO. 7 or at least 70% homology thereto, and (6) a gene that expresses iron oxidase having SEQ ID NO. 10 or at least 70% homology thereto.

In another aspect, the DNA construct is, from 5′ to 3′, the following genetic components in the following order: (1) a gene that expresses zinc oxidase having SEQ ID NO. 11 or at least 70% homology thereto, (2) a gene that expresses hexokinase having SEQ ID NO. 4 or at least 70% homology thereto, (3) a gene that expresses a heat shock protein having SEQ ID NO. 5 or at least 70% homology thereto, (4) a gene that expresses alcohol dehydrogenase having SEQ ID NO. 6 or at least 70% homology thereto, (5) an iron promoter having SEQ ID NO. 7 or at least 70% homology thereto, and (6) a gene that expresses fl having SEQ ID NO. 10 or at least 70% homology thereto.

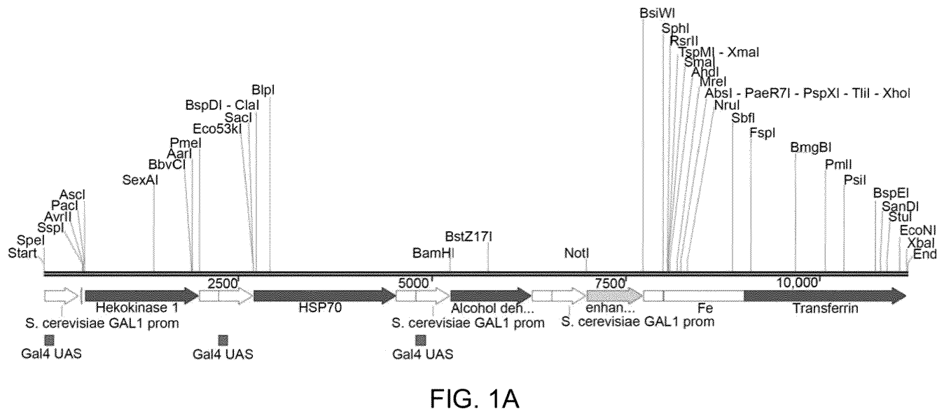

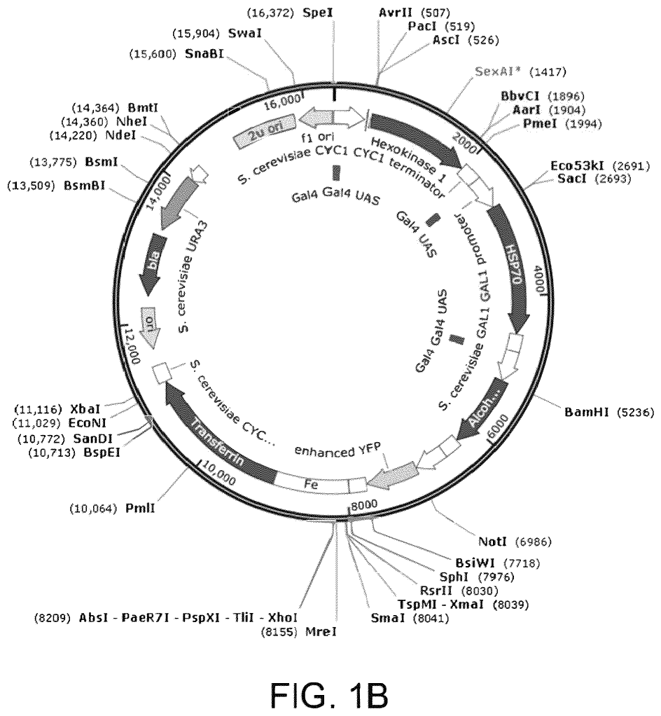

In another aspect, the DNA construct is, from 5′ to 3′, the following genetic components in the following order: (1) a GAL1 promoter, (2) a gene that expresses hexokinase having SEQ ID NO. 4 or at least 70% homology thereto, (3) a CYC1 terminator, (4) a GAL1 promoter, (5) a gene that expresses a heat shock protein having SEQ ID NO. 5 or at least 70% homology thereto, (6) a CYC1 terminator, (7) a GAL1 promoter, (8) a gene that expresses alcohol dehydrogenase having SEQ ID NO. 6 or at least 70% homology thereto, (9) a CYC1 terminator, (10) a GAL1 promoter, (11) a yellow fluorescent reporter protein having SEQ ID NO. 12 or at least 70% homology thereto, (12) a CYC1 terminator, (13) an iron promoter having SEQ ID NO. 7 or at least 70% homology thereto, and (14) a gene that expresses transferrin having SEQ ID NO. 8 or at least 70% homology thereto (FIG. 1). In another aspect, the DNA construct is SEQ ID NO. 1.

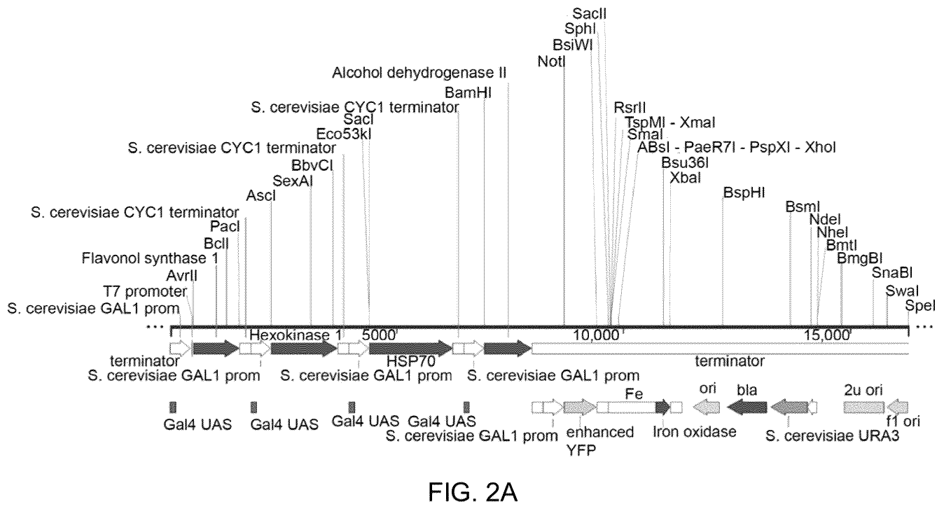

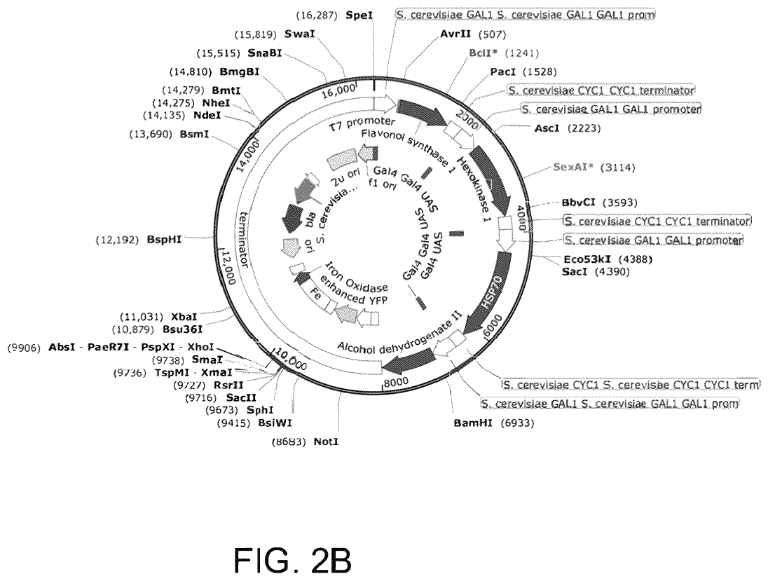

In another aspect, the DNA construct is, from 5′ to 3′, the following genetic components in the following order: (1) a GAL1 promoter, (2) a gene that expresses flavonol synthase having SEQ ID NO. 9 or at least 70% homology thereto, (3) a CYC1 terminator, (4) a GAL1 promoter, (5) a gene that expresses hexokinase having SEQ ID NO. 4 or at least 70% homology thereto, (6) a CYC1 terminator, (7) a GAL1 promoter, (8) a gene that expresses a heat shock protein having SEQ ID NO. 5 or at least 70% homology thereto, (9) a CYC1 terminator, (10) a GAL1 promoter, (11) a gene that expresses alcohol dehydrogenase having SEQ ID NO. 6 or at least 70% homology thereto, (12) a CYC1 terminator, (13) a GAL1 promoter, (14) a yellow fluorescent reporter protein having SEQ ID NO. 12 or at least 70% homology thereto, (15) a CYC1 terminator, (16) an iron promoter having SEQ ID NO. 7 or at least 70% homology thereto, and (17) a gene that expresses iron oxidase having SEQ ID NO. 10 or at least 70% homology thereto (FIG. 2). In another aspect, the DNA construct is SEQ ID NO. 2.

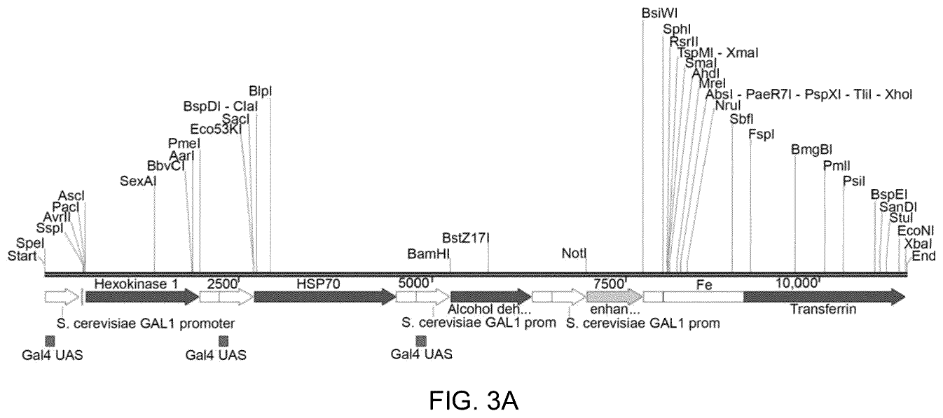

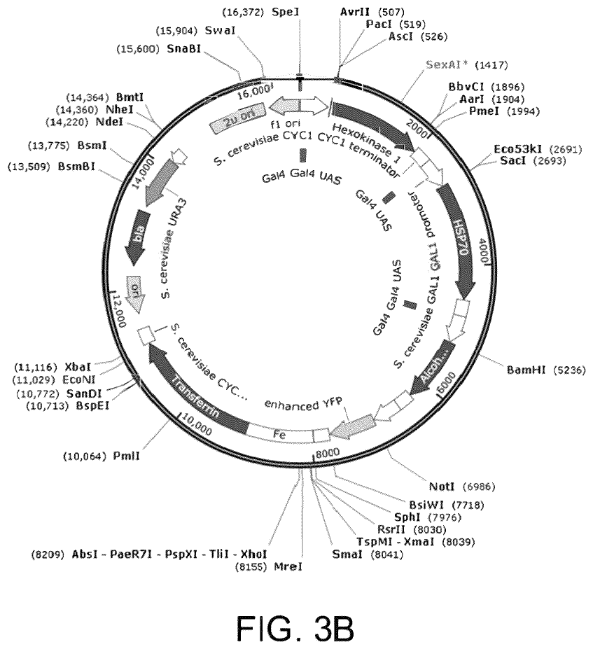

In another aspect, the DNA construct is, from 5′ to 3′, the following genetic components in the following order: (1) a GAL1 promoter, (2) a gene that expresses zinc oxidase having SEQ ID NO. 11 or at least 70% homology thereto, (3) a CYC1 terminator, (4) a GAL1 promoter, (5) a gene that expresses hexokinase having SEQ ID NO. 4 or at least 70% homology thereto, (6) a CYC1 terminator, (7) a GAL1 promoter, (8) a gene that expresses a heat shock protein having SEQ ID NO. 5 or at least 70% homology thereto, (9) a CYC1 terminator, (10) a GAL1 promoter, (11) a gene that expresses alcohol dehydrogenase having SEQ ID NO. 6 or at least 70% homology thereto, (12) a CYC1 terminator, (13) a GAL1 promoter, (14) a yellow fluorescent reporter protein having SEQ ID NO. 12 or at least 70% homology thereto, (15) a CYC1 terminator, (16) an iron promoter having SEQ ID NO. 7 or at least 70% homology thereto, and (17) a gene that expresses iron oxidase having SEQ ID NO. 10 or at least 70% homology thereto (FIG. 3). In another aspect, the DNA construct is SEQ ID NO. 3.

The DNA construct described herein can be part of a vector. In one aspect, the vector is a plasmid, a phagemid, a cosmid, a yeast artificial chromosome, a bacterial artificial chromosome, a virus, a phage, or a transposon.

In general, plasmid vectors containing replicon and control sequences that are derived from species compatible with the host cell are used in connection with these hosts. Vectors capable of high levels of expression of recombinant genes and proteins are well known in the art. The vector ordinarily carries a replication origin as well as marking sequences that are capable of providing phenotypic selection in transformed cells. Plasmid vectors useful for the transformation of a variety of host cells are well known and are commercially available. Such vectors include, but are not limited to, pWLNEO, pSV2CAT, pOG44, pXT1, pSG (Stratagene), pSVK3, pBSK, pBR322, pYES, pYES2, pBSKII, and pUC vectors.

Plasmids are double-stranded, autonomously-replicating, genetic elements that are not integrated into host cell chromosomes. Further, these genetic elements are usually not part of the host cell's central metabolism. In bacteria, plasmids may range from 1 kilobase (kb) to over 200 kb. Plasmids can be engineered to encode a number of useful traits including the production of secondary metabolites, antibiotic resistance, the production of useful proteins, degradation of complex molecules and/or environmental toxins, and others. Plasmids have been the subject of much research in the field of genetic engineering, as plasmids are convenient expression vectors for foreign DNA in, for example, microorganisms. Plasmids generally contain regulatory elements such as promoters and terminators and also usually have independent replication origins. Ideally, plasmids will be present in multiple copies per host cell and will contain selectable markers (such as genes for antibiotic resistance) to allow the ordinarily skilled artisan to select host cells that have been successfully transfected with the plasmids (for example, by culturing the host cells in a medium containing the antibiotic).

In one aspect, the vector encodes a selection marker. In a further aspect, the selection marker is a gene that confers resistance to an antibiotic. In certain aspects, during fermentation of host cells transformed with the vector, the cells are contacted with the antibiotic. For example, the antibiotic may be included in the culture medium. Cells that have not been successfully transformed cannot survive in the presence of the antibiotic; only cells containing the vector that confers antibiotic resistance can survive. Optionally, only cells containing the vector to be expressed will be cultured, as this will result in the highest production efficiency of the desired gene products (e.g., proteins having a UV-protective effect). Cells that do not contain the vector would otherwise compete with transformed cells for resources. In one aspect, the antibiotic is tetracycline, neomycin, kanamycin, ampicillin, hygromycin, chloramphenicol, amphotericin B, bacitracin, carbapenam, cephalosporin, ethambutol, fluoroquinolones, isonizid, methicillin, oxacillin, vancomycin, streptomycin, quinolines, rifampin, rifampicin, sulfonamides, cephalothin, erythromycin, streptomycin, gentamicin, penicillin, other commonly-used antibiotics, or a combination thereof.

In one aspect, when the vector is a plasmid, the plasmid can also contain a multiple cloning site or polylinker. In a further aspect, the polylinker contains recognition sites for multiple restriction enzymes. The polylinker can contain up to 2, 3, 4, 5, 6, 7, 8, 9, 10, 11, 12, 13, 14, 15, 16, 17, 18, 19, 20, or more than 20 recognition sites for restriction enzymes. Further, restriction sites may be added, disabled, or removed as required, using techniques known in the art. In one aspect, the plasmid contains restriction sites for any known restriction enzyme such as, for example, HindIII, KpnI, SacI, BamHI, BstXI, EcoRI, BsaBI, NotI, XhoI, SphI, SbaI, ApaI, SalI, ClaI, EcoRV, PstI, SmaI, XmaI, SpeI, EagI, SacII, or any combination thereof. In a further aspect, the plasmid contains more than one recognition site for the same restriction enzyme.

In one aspect, the restriction enzyme can cleave DNA at a palindromic or an asymmetrical restriction site. In a further aspect, the restriction enzyme cleaves DNA to leave blunt ends; in an alternative aspect, the restriction enzyme cleaves DNA to leave “sticky” or overhanging ends. In another aspect, the enzyme can cleave DNA a distance of from 20 bases to over 1000 bases away from the restriction site. A variety of restriction enzymes are commercially available and their recognition sequences, as well as instructions for use (e.g., amount of DNA needed, precise volumes of reagents, purification techniques, as well as information about salt concentration, pH, optimum temperature, incubation time, and the like) are made available by commercial enzyme suppliers.

In one aspect, a plasmid with a polylinker containing one or more restriction sites can be digested with one restriction enzyme and a nucleotide sequence of interest can be ligated into the plasmid using a commercially available DNA ligase enzyme. Several such enzymes are available, often as kits containing all reagents and instructions required for use. In another aspect, a plasmid with a polylinker containing two or more restriction sites can be simultaneously digested with two restriction enzymes and a nucleotide sequence of interest can be ligated into the plasmid using a DNA ligase enzyme. Using two restriction enzymes provides an asymmetric cut in the DNA, allowing for insertion of a nucleotide sequence of interest in a particular direction and/or on a particular strand of the double-stranded plasmid. Since RNA synthesis from a DNA template proceeds from 5′ to 3′, often starting just after a promoter, the order and direction of elements inserted into a plasmid is especially important. If a plasmid is to be simultaneously digested with multiple restriction enzymes, these enzymes must be compatible in terms of buffer, salt concentration, and other incubation parameters.

In some aspects, prior to ligation using a ligase enzyme, a plasmid that has been digested with a restriction enzyme is treated with an alkaline phosphatase enzyme to remove 5′ terminal phosphate groups. This prevents self-ligation of the plasmid and thus facilitates ligation of heterologous nucleic acid fragments into the plasmid.

In one aspect, the nucleic acids (e.g., genes that express hexokinase, alcohol dehydrogenase, and the like) used in the DNA constructs described herein can be amplified using the polymerase chain reaction (PCR) prior to being ligated into a plasmid or other vector. Typically, PCR-amplification techniques make use of primers or short, chemically-synthesized oligonucleotides that are complementary to regions on each respective strand flanking the DNA or nucleotide sequence to be amplified. A person having ordinary skill in the art will be able to design or choose primers based on the desired experimental conditions. In general, primers should be designed to provide for efficient and faithful replication of the target nucleic acids. Two primers are required for the amplification of each gene, one for the sense strand (that is, the strand containing the gene of interest) and one for the antisense strand (that is, the strand complementary to the gene of interest). Pairs of primers should have similar melting temperatures that are close to the PCR reaction's annealing temperature. In order to facilitate the PCR reaction, the following features should be avoided in primers: mononucleotide repeats, complementarity with other primers in the mixture, self-complementarity, and internal hairpins and/or loops. Methods of primer design are known in the art; additionally, computer programs exist that can assist the ordinarily skilled practitioner with primer design. Primers can optionally incorporate restriction enzyme recognition sites at their 5′ ends to assist in later ligation into plasmids or other vectors.

PCR can be carried out using purified DNA, unpurified DNA that has been integrated into a vector, or unpurified genomic DNA. The process for amplifying target DNA using PCR consists of introducing an excess of two primers having the characteristics described above to a mixture containing the sequence to be amplified, followed by a series of thermal cycles in the presence of a heat-tolerant or thermophilic DNA polymerase, such as, for example, any of Taq, Pfu, Pwo, Tfl, rTth, Tli, or Tma polymerases. A PCR “cycle” involves denaturation of the DNA through heating, followed by annealing of the primers to the target DNA, followed by extension of the primers using the thermophilic DNA polymerase and a supply of deoxynucleotide triphosphates (i.e., dCTP, dATP, dGTP, and TTP), along with buffers, salts, and other reagents as needed. In one aspect, the DNA segments created by primer extension during the PCR process can serve as templates for additional PCR cycles. Many PCR cycles can be performed to generate a large concentration of target DNA or gene. PCR can optionally be performed in a device or machine with programmable temperature cycles for denaturation, annealing, and extension steps. Further, PCR can be performed on multiple genes simultaneously in the same reaction vessel or microcentrifuge tube since the primers chosen will be specific to selected genes. PCR products can be purified by techniques known in the art such as, for example, gel electrophoresis followed by extraction from the gel using commercial kits and reagents.

In a further aspect, the vector can include an origin of replication, allowing it to use the host cell's replication machinery to create copies of itself.

As used herein, “operably linked” refers to the association of nucleic acid sequences on a single nucleic acid fragment so that the function of one affects the function of another. For example, if sequences for multiple genes are inserted into a single plasmid, their expression may be operably linked. Alternatively, a promoter is said to be operably linked with a coding sequence when it is capable of affecting the expression of the coding sequence.

As used herein, “expression” refers to transcription and/or accumulation of an mRNA derived from a gene or DNA fragment. Expression may also be used to refer to translation of mRNA into a peptide, polypeptide, or protein.

In one aspect, the gene that expresses hexokinase is isolated from yeast. In one aspect, the yeast species is, for example, Saccharomyces cerevisiae. In one aspect, the S. cerevisiae is a strain of yeast such as, for example, S288C, ySR127, YJM1355, YJM453, YJM1202, YJM326, YJM1526, YJM470, YJM456, YJM1387, YJM682, or another commonly-used strain. In a further aspect, the gene that expresses hexokinase has SEQ ID NO. 4 or at least 70% homology thereof, at least 75% homology thereof, at least 80% homology thereof, at least 85% homology thereof, at least 90% homology thereof, or at least 95% homology thereof. In one aspect, the gene that expresses hexokinase is isolated from Saccharomyces cerevisiae and can be found in GenBank with accession number M14410.1.

Other sequences expressing hexokinase or related or homologous genes can be identified in a database such as. for example. GenBank. In one aspect, sequences useful herein include those with the GI numbers listed in Table 1.

TABLE 1
Hexokinase Genes

| Source Organism | Sequence Description | GI Number |
|---|---|---|
| Saccharomyces cerevisiae | hexokinase PI | M14410.1 |
| Saccharomyces cerevisiae | chromosome VI sequence | CP020128.1 |
| Saccharomyces cerevisiae | chromosome VI sequence | CP014737.1 |
| Saccharomyces cerevisiae | chromosome VI sequence | CP011552.1 |
| Saccharomyces cerevisiae | hexokinase 1 | NM_001180018.3 |
| Saccharomyces cerevisiae | chromosome VI sequence | BK006940.2 |
| Saccharomyces cerevisiae | chromosome VI sequence | D50617.1 |
| Saccharomyces cerevisiae | hexokinase 1 | DQ332072.1 |
| Saccharomyces cerevisiae | chromosome VI sequence | CP004946.2 |
| Saccharomyces cerevisiae | chromosome VI sequence | CP008547.1 |
| Saccharomyces cerevisiae | chromosome VI sequence | CP008530.1 |
| Saccharomyces cerevisiae | chromosome VI sequence | CP007901.1 |
| Saccharomyces cerevisiae | chromosome VI sequence | CP004902.2 |
| Saccharomyces cerevisiae | chromosome VI sequence | CP004929.2 |
| Saccharomyces cerevisiae | chromosome VI sequence | CP004898.2 |
| Saccharomyces cerevisiae | chromosome VI sequence | CP008462.1 |
| Saccharomyces cerevisiae | chromosome VI sequence | CP004975.2 |
| Saccharomyces cerevisiae | chromosome VI sequence | CP004904.2 |
| Saccharomyces cerevisiae | chromosome VI sequence | CP004903.2 |
| Saccharomyces cerevisiae | chromosome VI sequence | CP004952.2 |
| Saccharomyces cerevisiae | chromosome VI sequence | CP004910.2 |
| Saccharomyces cerevisiae | hexokinase isoenzyme 1 | JF898945.1 |
| Saccharomyces cerevisiae | chromosome VI sequence | CP008105.1 |
| Saccharomyces cerevisiae | chromosome VI sequence | CP008071.1 |
| Saccharomyces cerevisiae | chromosome VI sequence | CP004909.2 |
| Saccharomyces cerevisiae | chromosome VI sequence | CP004927.2 |
| Saccharomyces cerevisiae | chromosome VI sequence | CP008241.1 |
| Saccharomyces cerevisiae | chromosome VI sequence | CP008292.1 |
| Saccharomyces cerevisiae | chromosome VI sequence | CP008275.1 |
| Saccharomyces cerevisiae | chromosome VI sequence | CP008224.1 |
| Saccharomyces cerevisiae | chromosome VI sequence | CP008411.1 |
| Saccharomyces cerevisiae | chromosome VI sequence | CP008377.1 |
| Saccharomyces cerevisiae | chromosome VI sequence | CP008428.1 |
| Saccharomyces cerevisiae | chromosome VI sequence | CP008581.1 |
| Saccharomyces cerevisiae | chromosome VI sequence | CP008598.1 |
| Saccharomyces cerevisiae | chromosome VI sequence | CP008173.1 |
| Saccharomyces cerevisiae | chromosome VI sequence | CP008156.1 |
| Saccharomyces cerevisiae | chromosome VI sequence | CP008683.1 |
| Saccharomyces cerevisiae | chromosome VI sequence | CP008088.1 |
| Saccharomyces cerevisiae | chromosome VI sequence | CP008020.1 |
| Saccharomyces cerevisiae | chromosome VI sequence | CP007986.1 |
| Saccharomyces cerevisiae | chromosome VI sequence | CP007969.1 |
| Saccharomyces cerevisiae | chromosome VI sequence | CP007952.1 |
| Saccharomyces cerevisiae | chromosome VI sequence | CP007918.1 |
| Saccharomyces cerevisiae | chromosome VI sequence | CP007884.1 |
| Saccharomyces cerevisiae | chromosome VI sequence | CP007867.1 |
| Saccharomyces cerevisiae | chromosome VI sequence | CP004925.2 |
| Saccharomyces cerevisiae | chromosome VI sequence | CP004934.2 |
| Saccharomyces cerevisiae | chromosome VI sequence | CP004913.2 |
| Saccharomyces cerevisiae | chromosome VI sequence | CP004893.2 |
| Saccharomyces cerevisiae | chromosome VI sequence | CP004951.2 |
| Saccharomyces cerevisiae | chromosome VI sequence | CP004890.2 |
| Saccharomyces cerevisiae | chromosome VI sequence | CP004949.1 |
| Saccharomyces cerevisiae | chromosome VI sequence | CP004948.1 |
| Saccharomyces cerevisiae | chromosome VI sequence | CP004940.1 |
| Saccharomyces cerevisiae | hexokinase isoenzyme 1 | JF898949.1 |
| Saccharomyces cerevisiae | hexokinase isoenzyme 1 | JF898946.1 |
| Saccharomyces cerevisiae | chromosome VI sequence | CP008258.1 |
| Saccharomyces cerevisiae | chromosome VI sequence | CP008394.1 |
| Saccharomyces cerevisiae | chromosome VI sequence | CP008496.1 |
| Saccharomyces cerevisiae | chromosome VI sequence | CP008445.1 |
| Saccharomyces cerevisiae | chromosome VI sequence | CP008513.1 |
| Saccharomyces cerevisiae | hexokinase isoenzyme 1 | JF898948.1 |
| Saccharomyces cerevisiae | chromosome VI sequence | CP020162.1 |
| Saccharomyces cerevisiae | chromosome VI sequence | CP004979.2 |
| Saccharomyces cerevisiae | chromosome VI sequence | CP004919.2 |
| Saccharomyces cerevisiae | chromosome VI sequence | CP004918.2 |
| Saccharomyces cerevisiae | chromosome VI sequence | CP004908.2 |
| Saccharomyces cerevisiae | chromosome VI sequence | CP004917.2 |
| Saccharomyces cerevisiae | chromosome VI sequence | CP004907.2 |
| Saccharomyces cerevisiae | chromosome VI sequence | CP004897.2 |
| Saccharomyces cerevisiae | chromosome VI sequence | CP004916.2 |
| Saccharomyces cerevisiae | chromosome VI sequence | CP004906.2 |
| Saccharomyces cerevisiae | chromosome VI sequence | CP004896.2 |
| Saccharomyces cerevisiae | chromosome VI sequence | CP008326.1 |
| Saccharomyces cerevisiae | chromosome VI sequence | CP008309.1 |
| Saccharomyces cerevisiae | chromosome VI sequence | CP008360.1 |
| Saccharomyces cerevisiae | chromosome VI sequence | CP008343.1 |
| Saccharomyces cerevisiae | chromosome VI sequence | CP008479.1 |
| Saccharomyces cerevisiae | chromosome VI sequence | CP008564.1 |
| Saccharomyces cerevisiae | chromosome VI sequence | CP008666.1 |
| Saccharomyces cerevisiae | chromosome VI sequence | CP008649.1 |
| Saccharomyces cerevisiae | chromosome VI sequence | CP008615.1 |
| Saccharomyces cerevisiae | chromosome VI sequence | CP008122.1 |
| Saccharomyces cerevisiae | chromosome VI sequence | CP008054.1 |
| Saccharomyces cerevisiae | chromosome VI sequence | CP008037.1 |
| Saccharomyces cerevisiae | chromosome VI sequence | CP007935.1 |
| Saccharomyces cerevisiae | chromosome VI sequence | CP007850.1 |
| Saccharomyces cerevisiae | chromosome VI sequence | CP007816.1 |
| Saccharomyces cerevisiae | chromosome VI sequence | CP004915.2 |
| Saccharomyces cerevisiae | chromosome VI sequence | CP004905.2 |
| Saccharomyces cerevisiae | chromosome VI sequence | CP004944.2 |
| Saccharomyces cerevisiae | chromosome VI sequence | CP004914.2 |
| Saccharomyces cerevisiae | chromosome VI sequence | CP004963.2 |
| Saccharomyces cerevisiae | chromosome VI sequence | CP004923.2 |
| Saccharomyces cerevisiae | chromosome VI sequence | CP004932.2 |
| Saccharomyces cerevisiae | chromosome VI sequence | CP004922.2 |
| Saccharomyces cerevisiae | chromosome VI sequence | CP004972.2 |
| Saccharomyces cerevisiae | chromosome VI sequence | CP004931.2 |
| Saccharomyces cerevisiae | chromosome VI sequence | CP007833.1 |

In one aspect, the gene that expresses a heat shock protein is isolated from yeast. In one aspect, the yeast species is, for example, Saccharomyces cerevisiae. In one aspect, the S. cerevisiae is a strain of yeast such as, for example, S288C, ySR127, YJM1355, YJM453, YJM1202, YJM326, YJM1526, YJM470, YJM456, YJM1387, YJM682, or another commonly-used strain. In a further aspect, the gene that expresses a heat shock protein is HSP70 and has SEQ ID NO. 5 or at least 70% homology thereof, at least 75% homology thereof, at least 80% homology thereof, at least 85% homology thereof, at least 90% homology thereof, or at least 95% homology thereof. In one aspect, the gene that expresses a heat shock protein is isolated from Saccharomyces cerevisiae and can be found in GenBank with accession number X13713.

Other sequences expressing heat shock proteins or related or homologous genes can be identified in a database such as, for example, GenBank. In one aspect, sequences useful herein include those with the GI numbers listed in Table 2.

TABLE 2
Heat Shock Protein Genes

| Source Organism | Sequence Description | GI Number |
|---|---|---|
| Saccharomyces cerevisiae | chromosome IV sequence | CP020126.1 |
| Saccharomyces cerevisiae | chromosome IV sequence | CP004710.2 |
| Saccharomyces cerevisiae | chromosome IV sequence | CP011550.1 |
| Saccharomyces cerevisiae | HSP70 family ATPase SSB1 | BK006938.2 |
| Saccharomyces cerevisiae | chromosome IV sequence | NM_001180289.1 |
| Saccharomyces cerevisiae | chromosome IV sequence | FN393064.1 |
| Saccharomyces cerevisiae | SSB1 heat shock cognate gene | Z74277.1 |
| Saccharomyces cerevisiae | chromosome IV sequence | X13713.1 |
| Saccharomyces cerevisiae | chromosome IV sequence | EF058944.1 |
| Saccharomyces cerevisiae | chromosome IV sequence | CP020228.1 |
| Saccharomyces cerevisiae | chromosome IV sequence | CP020160.1 |
| Saccharomyces cerevisiae | chromosome IV sequence | CP004738.2 |
| Saccharomyces cerevisiae | chromosome IV sequence | CP004688.2 |
| Saccharomyces cerevisiae | chromosome IV sequence | CP004678.2 |
| Saccharomyces cerevisiae | chromosome IV sequence | CP004727.2 |
| Saccharomyces cerevisiae | chromosome IV sequence | CP004717.2 |
| Saccharomyces cerevisiae | chromosome IV sequence | CP004687.2 |
| Saccharomyces cerevisiae | chromosome IV sequence | CP004667.2 |
| Saccharomyces cerevisiae | chromosome IV sequence | CP004746.2 |
| Saccharomyces cerevisiae | chromosome IV sequence | CP004716.2 |
| Saccharomyces cerevisiae | chromosome IV sequence | CP004676.2 |
| Saccharomyces cerevisiae | chromosome IV sequence | CP008239.1 |
| Saccharomyces cerevisiae | chromosome IV sequence | CP008324.1 |
| Saccharomyces cerevisiae | chromosome IV sequence | CP008273.1 |
| Saccharomyces cerevisiae | chromosome IV sequence | CP008256.1 |
| Saccharomyces cerevisiae | chromosome IV sequence | CP008222.1 |
| Saccharomyces cerevisiae | chromosome IV sequence | CP008409.1 |
| Saccharomyces cerevisiae | chromosome IV sequence | CP008392.1 |
| Saccharomyces cerevisiae | chromosome IV sequence | CP008375.1 |
| Saccharomyces cerevisiae | chromosome IV sequence | CP008358.1 |
| Saccharomyces cerevisiae | chromosome IV sequence | CP008341.1 |
| Saccharomyces cerevisiae | chromosome IV sequence | CP008494.1 |
| Saccharomyces cerevisiae | chromosome IV sequence | CP008443.1 |
| Saccharomyces cerevisiae | chromosome IV sequence | CP008579.1 |
| Saccharomyces cerevisiae | chromosome IV sequence | CP008511.1 |
| Saccharomyces cerevisiae | chromosome IV sequence | CP008647.1 |
| Saccharomyces cerevisiae | chromosome IV sequence | CP008630.1 |
| Saccharomyces cerevisiae | chromosome IV sequence | CP008596.1 |
| Saccharomyces cerevisiae | chromosome IV sequence | CP008188.1 |
| Saccharomyces cerevisiae | chromosome IV sequence | CP008171.1 |
| Saccharomyces cerevisiae | chromosome IV sequence | CP008154.1 |
| Saccharomyces cerevisiae | chromosome IV sequence | CP008681.1 |
| Saccharomyces cerevisiae | chromosome IV sequence | CP008137.1 |
| Saccharomyces cerevisiae | chromosome IV sequence | CP008120.1 |
| Saccharomyces cerevisiae | chromosome IV sequence | CP008086.1 |
| Saccharomyces cerevisiae | chromosome IV sequence | CP008052.1 |
| Saccharomyces cerevisiae | chromosome IV sequence | CP008035.1 |
| Saccharomyces cerevisiae | chromosome IV sequence | CP008001.1 |
| Saccharomyces cerevisiae | chromosome IV sequence | CP007984.1 |
| Saccharomyces cerevisiae | chromosome IV sequence | CP007950.1 |
| Saccharomyces cerevisiae | chromosome IV sequence | CP007899.1 |
| Saccharomyces cerevisiae | chromosome IV sequence | CP007882.1 |
| Saccharomyces cerevisiae | chromosome IV sequence | CP007831.1 |
| Saccharomyces cerevisiae | chromosome IV sequence | CP004745.2 |
| Saccharomyces cerevisiae | chromosome IV sequence | CP004684.2 |
| Saccharomyces cerevisiae | chromosome IV sequence | CP004743.2 |
| Saccharomyces cerevisiae | chromosome IV sequence | CP004713.2 |
| Saccharomyces cerevisiae | chromosome IV sequence | CP004692.2 |
| Saccharomyces cerevisiae | chromosome IV sequence | CP004742.2 |
| Saccharomyces cerevisiae | chromosome IV sequence | CP004722.2 |
| Saccharomyces cerevisiae | chromosome IV sequence | CP004672.2 |
| Saccharomyces cerevisiae | chromosome IV sequence | CP004701.2 |
| Saccharomyces cerevisiae | chromosome IV sequence | CP004681.2 |
| Saccharomyces cerevisiae | chromosome IV sequence | CP004690.2 |
| Saccharomyces cerevisiae | chromosome IV sequence | CP004670.2 |
| Saccharomyces cerevisiae | chromosome IV sequence | CP011082.1 |
| Saccharomyces cerevisiae | chromosome IV sequence | CP004729.1 |
| Saccharomyces cerevisiae | heat shock protein 70 | M25395.1 |
| Saccharomyces cerevisiae | chromosome IV sequence | CP020211.1 |
| Saccharomyces cerevisiae | chromosome IV sequence | CP004748.2 |
| Saccharomyces cerevisiae | chromosome IV sequence | CP004697.2 |
| Saccharomyces cerevisiae | chromosome IV sequence | CP004677.2 |
| Saccharomyces cerevisiae | chromosome IV sequence | CP004726.2 |
| Saccharomyces cerevisiae | chromosome IV sequence | CP004706.2 |
| Saccharomyces cerevisiae | chromosome IV sequence | CP008307.1 |
| Saccharomyces cerevisiae | chromosome IV sequence | CP008290.1 |
| Saccharomyces cerevisiae | chromosome IV sequence | CP008477.1 |
| Saccharomyces cerevisiae | chromosome IV sequence | CP008460.1 |
| Saccharomyces cerevisiae | chromosome IV sequence | CP008426.1 |
| Saccharomyces cerevisiae | chromosome IV sequence | CP008562.1 |
| Saccharomyces cerevisiae | chromosome IV sequence | CP008664.1 |
| Saccharomyces cerevisiae | chromosome IV sequence | CP008613.1 |
| Saccharomyces cerevisiae | chromosome IV sequence | CP008103.1 |
| Saccharomyces cerevisiae | chromosome IV sequence | CP008069.1 |
| Saccharomyces cerevisiae | chromosome IV sequence | CP008018.1 |
| Saccharomyces cerevisiae | chromosome IV sequence | CP007967.1 |
| Saccharomyces cerevisiae | chromosome IV sequence | CP007933.1 |
| Saccharomyces cerevisiae | chromosome IV sequence | CP007916.1 |
| Saccharomyces cerevisiae | chromosome IV sequence | CP007865.1 |
| Saccharomyces cerevisiae | chromosome IV sequence | CP007848.1 |
| Saccharomyces cerevisiae | chromosome IV sequence | CP007814.1 |
| Saccharomyces cerevisiae | chromosome IV sequence | CP004695.2 |
| Saccharomyces cerevisiae | chromosome IV sequence | CP004675.2 |
| Saccharomyces cerevisiae | chromosome IV sequence | CP004744.2 |
| Saccharomyces cerevisiae | chromosome IV sequence | CP004724.2 |
| Saccharomyces cerevisiae | chromosome IV sequence | CP004714.2 |
| Saccharomyces cerevisiae | chromosome IV sequence | CP004704.2 |
| Saccharomyces cerevisiae | chromosome IV sequence | CP004674.2 |
| Saccharomyces cerevisiae | chromosome IV sequence | CP004733.2 |
| Saccharomyces cerevisiae | chromosome IV sequence | CP008205.1 |

In one aspect, the gene that expresses alcohol dehydrogenase is isolated from yeast. In one aspect, the yeast species is, for example, Saccharomyces cerevisiae. In one aspect, the S. cerevisiae is a strain of yeast such as, for example, S288C, ySR127,YJM1355, YJM453, YJM1202, YJM326, YJM1526, YJM470, YJM456, YJM1387,YJM682, or another commonly-used strain. In a further aspect, the gene that expresses alcohol dehydrogenase has SEQ ID NO. 6 or at least 70% homology thereof, at least 75% homology thereof, at least 80% homology thereof, at least 85% homology thereof, at least 90% homology thereof, or at least 95% homology thereof.

In one aspect, the gene that expresses alcohol dehydrogenase is isolated from Saccharomyces cerevisiae and can be found in GenBank with GI number J01314.1.

Other sequences expressing alcohol dehydrogenase or related or homologous genes can be identified in a database such as, for example, GenBank. In one aspect, sequences useful herein include those with the GI numbers listed in Table 3.

TABLE 3
Alcohol Dehydrogenase Genes

| Source Organism | Sequence Description | GI Number |
|---|---|---|
| Saccharomyces cerevisiae | alcohol dehydrogenase II | J01314.1 |
| | gene | |
| Saccharomyces cerevisiae | chromosome XIII sequence | CP005453.2 |
| Saccharomyces cerevisiae | chromosome XIII sequence | CP020135.1 |
| Saccharomyces cerevisiae | chromosome XIII sequence | CP005452.2 |
| Saccharomyces cerevisiae | chromosome XIII sequence | CP005450.2 |
| Saccharomyces cerevisiae | chromosome XIII sequence | BK006946.2 |
| Saccharomyces cerevisiae | alcohol dehydrogenase II | NM_001182812.1 |
| | gene | |
| Saccharomyces cerevisiae | alcohol dehydrogenase II | EF059086.1 |
| | gene | |
| Saccharomyces cerevisiae | chromosome XIII sequence | Z49212.1 |
| Saccharomyces cerevisiae | glucose-repressible ADH2 | KJ137139.1 |
| Saccharomyces cerevisiae | alcohol dehydrogenase II | M38457.1 |
| | gene | |
| Saccharomyces cerevisiae | glucose-repressible ADH2 | KJ137141.1 |
| Saccharomyces cerevisiae | glucose-repressible ADH2 | KJ137132.1 |
| Saccharomyces cerevisiae | chromosome XIII sequence | CP005464.2 |
| Saccharomyces cerevisiae | chromosome XIII sequence | CP005483.2 |
| Saccharomyces cerevisiae | chromosome XIII sequence | CP005432.2 |
| Saccharomyces cerevisiae | chromosome XIII sequence | CP020203.1 |
| Saccharomyces cerevisiae | chromosome XIII sequence | CP005482.2 |
| Saccharomyces cerevisiae | chromosome XIII sequence | CP005472.2 |
| Saccharomyces cerevisiae | chromosome XIII sequence | LN907796.1 |
| Saccharomyces cerevisiae | chromosome XIII sequence | CP005456.1 |
| Saccharomyces cerevisiae | chromosome XIII sequence | CP005455.1 |
| Saccharomyces cerevisiae | chromosome XIII sequence | CP005440.1 |
| Saccharomyces cerevisiae | chromosome XIII sequence | CP005465.2 |
| Saccharomyces cerevisiae | chromosome XIII sequence | CP005405.2 |
| Saccharomyces cerevisiae | chromosome XIII sequence | CP005414.2 |
| Saccharomyces cerevisiae | chromosome XIII sequence | CP005403.2 |
| Saccharomyces cerevisiae | chromosome XIII sequence | CP005412.2 |
| Saccharomyces cerevisiae | chromosome XIII sequence | CP011559.1 |
| Saccharomyces cerevisiae | chromosome XIII sequence | CP005426.1 |
| Saccharomyces cerevisiae | chromosome XIII sequence | CP005406.1 |
| Saccharomyces cerevisiae | alcohol dehydrogenase II | JX901290.1 |
| | gene | |
| Saccharomyces cerevisiae | chromosome XIII sequence | CP005451.2 |
| Saccharomyces cerevisiae | chromosome XIII sequence | CP005436.1 |
| Saccharomyces cerevisiae | glucose-repressible ADH2 | KJ137135.1 |
| Saccharomyces cerevisiae | chromosome XIII sequence | CP008010.1 |
| Saccharomyces cerevisiae | chromosome XIII sequence | CP020169.1 |
| Saccharomyces cerevisiae | chromosome XIII sequence | CP005449.2 |
| Saccharomyces cerevisiae | chromosome XIII sequence | CP005429.2 |
| Saccharomyces cerevisiae | chromosome XIII sequence | CP005419.2 |
| Saccharomyces cerevisiae | chromosome XIII sequence | CP005409.2 |
| Saccharomyces cerevisiae | chromosome XIII sequence | CP005428.2 |
| Saccharomyces cerevisiae | chromosome XIII sequence | CP005418.2 |
| Saccharomyces cerevisiae | chromosome XIII sequence | CP005408.2 |
| Saccharomyces cerevisiae | chromosome XIII sequence | CP005477.2 |
| Saccharomyces cerevisiae | chromosome XIII sequence | CP005417.2 |
| Saccharomyces cerevisiae | chromosome XIII sequence | CP005425.2 |
| Saccharomyces cerevisiae | chromosome XIII sequence | CP008265.1 |
| Saccharomyces cerevisiae | chromosome XIII sequence | CP008367.1 |
| Saccharomyces cerevisiae | chromosome XIII sequence | CP008554.1 |
| Saccharomyces cerevisiae | chromosome XIII sequence | CP008537.1 |
| Saccharomyces cerevisiae | chromosome XIII sequence | CP008520.1 |
| Saccharomyces cerevisiae | chromosome XIII sequence | CP008129.1 |
| Saccharomyces cerevisiae | chromosome XIII sequence | CP007993.1 |
| Saccharomyces cerevisiae | chromosome XIII sequence | CP005444.2 |
| Saccharomyces cerevisiae | chromosome XIII sequence | CP005434.2 |
| Saccharomyces cerevisiae | chromosome XIII sequence | CP005424.2 |
| Saccharomyces cerevisiae | chromosome XIII sequence | CP005404.2 |
| Saccharomyces cerevisiae | chromosome XIII sequence | CP005423.2 |
| Saccharomyces cerevisiae | chromosome XIII sequence | CP005422.2 |
| Saccharomyces cerevisiae | chromosome XIII sequence | CP005402.2 |
| Saccharomyces cerevisiae | chromosome XIII sequence | CP005421.2 |
| Saccharomyces cerevisiae | chromosome XIII sequence | CP005411.2 |
| Saccharomyces cerevisiae | chromosome XIII sequence | CP005420.2 |
| Saccharomyces cerevisiae | chromosome XIII sequence | CP005427.1 |
| Saccharomyces cerevisiae | chromosome XIII sequence | CP005416.1 |
| Saccharomyces cerevisiae | glucose-repressible ADH2 | KJ137136.1 |
| Saccharomyces cerevisiae | glucose-repressible ADH2 | KJ137134.1 |
| Saccharomyces cerevisiae | glucose-repressible ADH2 | KJ137133.1 |
| Saccharomyces cerevisiae | glucose-repressible ADH2 | KJ137142.1 |
| Saccharomyces cerevisiae | glucose-repressible ADH2 | KJ137138.1 |
| Saccharomyces cerevisiae | chromosome XIII sequence | CP005475.2 |
| Saccharomyces cerevisiae | chromosome XIII sequence | CP008401.1 |
| Saccharomyces cerevisiae | chromosome XIII sequence | CP008503.1 |
| Saccharomyces cerevisiae | chromosome XIII sequence | CP005398.2 |
| Saccharomyces cerevisiae | chromosome XIII sequence | CP005478.2 |
| Saccharomyces cerevisiae | chromosome XIII sequence | CP005437.2 |
| Saccharomyces cerevisiae | chromosome XIII sequence | CP005407.2 |
| Saccharomyces cerevisiae | chromosome XIII sequence | CP005454.2 |
| Saccharomyces cerevisiae | chromosome XIII sequence | CP005462.2 |
| Saccharomyces cerevisiae | chromosome XIII sequence | CP005461.2 |
| Saccharomyces cerevisiae | chromosome XIII sequence | CP005401.2 |
| Saccharomyces cerevisiae | chromosome XIII sequence | CP005396.1 |
| Saccharomyces cerevisiae | chromosome XIII sequence | CP005479.2 |
| Saccharomyces cerevisiae | chromosome XIII sequence | CP005469.2 |
| Saccharomyces cerevisiae | chromosome XIII sequence | CP005399.2 |
| Saccharomyces cerevisiae | chromosome XIII sequence | CP005397.2 |
| Saccharomyces cerevisiae | chromosome XIII sequence | CP005415.2 |
| Saccharomyces cerevisiae | chromosome XIII sequence | CP005395.2 |
| Saccharomyces cerevisiae | chromosome XIII sequence | CP008248.1 |
| Saccharomyces cerevisiae | chromosome XIII sequence | CP008333.1 |
| Saccharomyces cerevisiae | chromosome XIII sequence | CP008316.1 |
| Saccharomyces cerevisiae | chromosome XIII sequence | CP008299.1 |
| Saccharomyces cerevisiae | chromosome XIII sequence | CP008282.1 |
| Saccharomyces cerevisiae | chromosome XIII sequence | CP008231.1 |
| Saccharomyces cerevisiae | chromosome XIII sequence | CP008418.1 |
| Saccharomyces cerevisiae | chromosome XIII sequence | CP008384.1 |
| Saccharomyces cerevisiae | chromosome XIII sequence | CP008350.1 |
| Saccharomyces cerevisiae | chromosome XIII sequence | CP008486.1 |
| Saccharomyces cerevisiae | chromosome XIII sequence | CP008469.1 |

In one aspect, the gene that expresses an iron promoter is isolated from a bacterium. In one aspect, the bacterium species is a Mycobacterium species such as, for example, M. avium, M. yongonense, M. chimaera, M. intracellulare, M. kansasii, M. marinum, M. ulcerans, or M. tuberculosis. In a further aspect, the gene that expresses iron promoter has SEQ ID NO. 7 or at least 70% homology thereof, at least 75% homology thereof, at least 80% homology thereof, at least 85% homology thereof, at least 90% homology thereof, or at least 95% homology thereof. In one aspect, the gene that expresses an iron promoter is isolated from Mycobacterium avium subsp. paratuberculosis MAP4 and can be found in GenBank with GI number CP005928.

Other sequences expressing an iron promoter or related or homologous genes can be identified in a database such as, for example, GenBank. In one aspect, sequences useful herein include those with the GI numbers listed in Table 4.

TABLE 4
Iron Promoter Genes

| Source Organism | Sequence Description | GI Number |
|---|---|---|
| Mycobacterium avium subsp. | genomic DNA | CP015495.1 |
| paratuberculosis | | |
| Mycobacterium avium subsp. | genomic DNA | CP010114.1 |
| paratuberculosis | | |
| Mycobacterium avium subsp. | genomic DNA | CP010113.1 |
| paratuberculosis | | |
| Mycobacterium avium subsp. | genomic DNA | CP005928.1 |
| paratuberculosis | | |
| Mycobacterium avium subsp. | genomic DNA | AF280067.2 |
| paratuberculosis | | |
| Mycobacterium avium subsp. | genomic DNA | AE016958.1 |
| paratuberculosis | | |
| Mycobacterium avium subsp. | genomic DNA | AP012555.1 |
| hominissuis | | |
| Mycobacterium avium subsp. | | CP018363.1 |
| hominissuis | | |
| Mycobacterium avium subsp. avium | genomic DNA | CP009614.1 |
| Mycobacterium avium subsp. avium | genomic DNA | CP009482.1 |
| Mycobacterium avium subsp. avium | genomic DNA | CP009493.1 |
| Mycobacterium avium | genomic DNA | CP000479.1 |
| Mycobacterium avium | genomic DNA | CP016396.1 |
| Mycobacterium avium | genomic DNA | D78144.1 |
| Mycobacterium avium subsp. | genomic DNA | AJ584651.1 |
| paratuberculosis | | |
| Mycobacterium avium subsp. avium | genomic DNA | AB325677.1 |
| Mycobacterium colombiense | genomic DNA | CP020821.1 |
| Mycobacterium chimaera | genomic DNA | CP019221.1 |
| Mycobacterium yongonense | genomic DNA | CP015965.1 |
| Mycobacterium yongonense | genomic DNA | CP015964.1 |
| Mycobacterium chimaera | genomic DNA | LT703505.1 |
| Mycobacterium chimaera | genomic DNA | CP012885.2 |
| Mycobacterium intracellulare | genomic DNA | CP009499.1 |
| Mycobacterium sp. 05-1390 | genomic DNA | CP003347.1 |
| Mycobacterium indicus pranii | genomic DNA | CP002275.1 |
| Mycobacterium sp. MOTT36Y | genomic DNA | CP003491.1 |
| Mycobacterium intracellulare | genomic DNA | CP003324.1 |
| Mycobacterium intracellulare | genomic DNA | CP003323.1 |
| Mycobacterium intracellulare | genomic DNA | CP003322.1 |
| Mycobacterium simiae | genomic DNA | CP010996.1 |
| Mycobacterium shigaense | genomic DNA | AP018164.1 |
| Mycobacterium gordonae | genomic DNA | Y10378.1 |
| Mycobacterium kansasii | genomic DNA | CP019888.1 |
| Mycobacterium kansasii | genomic DNA | CP019887.1 |
| Mycobacterium kansasii | genomic DNA | CP019886.1 |
| Mycobacterium kansasii | genomic DNA | CP019885.1 |
| Mycobacterium kansasii | genomic DNA | CP019884.1 |
| Mycobacterium kansasii | genomic DNA | CP019883.1 |
| Mycobacterium kansasii | genomic DNA | CP009483.1 |
| Mycobacterium kansasii | genomic DNA | CP006835.1 |
| Mycobacterium marinum | genomic DNA | AB716948.1 |
| Mycobacterium marinum | genomic DNA | HG917972.2 |
| Mycobacterium marinum | genomic DNA | CP000854.1 |
| Mycobacterium marinum | genomic DNA | AB716942.1 |
| Mycobacterium ulcerans | genomic DNA | AP017635.1 |
| Mycobacterium ulcerans | genomic DNA | AP017624.1 |
| Mycobacterium liflandii | genomic DNA | CP003899.1 |
| Mycobacterium marinum | genomic DNA | AY225215.1 |
| Mycobacterium ulcerans | genomic DNA | CP000325.1 |
| Mycobacterium sp. 012931 | genomic DNA | AB716954.1 |
| Mycobacterium ulcerans | genomic DNA | AJ300576.1 |
| Mycobacterium haemophilum | genomic DNA | CP011883.2 |
| Mycobacterium tuberculosis | genomic DNA | CP010340.1 |
| Mycobacterium tuberculosis | genomic DNA | CP005386.1 |
| Mycobacterium canettii | genomic DNA | FO203510.1 |
| Mycobacterium canettii | genomic DNA | FO203508.1 |
| Mycobacterium tuberculosis | genomic DNA | GQ150314.1 |
| Mycobacterium tuberculosis | genomic DNA | AP018036.1 |
| Mycobacterium tuberculosis | genomic DNA | AP018035.1 |
| Mycobacterium tuberculosis | genomic DNA | AP018034.1 |
| Mycobacterium tuberculosis | genomic DNA | CP017598.1 |
| Mycobacterium tuberculosis | genomic DNA | CP017597.1 |
| Mycobacterium tuberculosis | genomic DNA | CP017596.1 |
| Mycobacterium tuberculosis | genomic DNA | CP017595.1 |
| Mycobacterium tuberculosis | genomic DNA | CP017594.1 |
| Mycobacterium tuberculosis | genomic DNA | CP017593.1 |
| Mycobacterium tuberculosis | genomic DNA | AP018033.1 |
| Mycobacterium tuberculosis | genomic DNA | CP020381.2 |
| Mycobacterium tuberculosis | genomic DNA | CP009195.1 |
| Mycobacterium tuberculosis | genomic DNA | CP009187.1 |
| Mycobacterium tuberculosis | genomic DNA | CP009186.1 |
| Mycobacterium tuberculosis | genomic DNA | CP009183.1 |
| Mycobacterium tuberculosis | genomic DNA | CP009206.1 |
| Mycobacterium tuberculosis | genomic DNA | CP009199.1 |
| Mycobacterium tuberculosis | genomic DNA | CP009202.1 |
| Mycobacterium tuberculosis | genomic DNA | CP009193.1 |
| Mycobacterium tuberculosis | genomic DNA | CP009192.1 |
| Mycobacterium tuberculosis | genomic DNA | CP009191.1 |
| Mycobacterium tuberculosis | genomic DNA | CP009190.1 |
| Mycobacterium tuberculosis | genomic DNA | CP009197.1 |
| Mycobacterium tuberculosis | genomic DNA | CP009196.1 |
| Mycobacterium tuberculosis | genomic DNA | CP009194.1 |
| Mycobacterium tuberculosis | genomic DNA | CP009189.1 |
| Mycobacterium tuberculosis | genomic DNA | CP009188.1 |
| Mycobacterium tuberculosis | genomic DNA | CP009185.1 |
| Mycobacterium tuberculosis | genomic DNA | CP009184.1 |
| Mycobacterium tuberculosis | genomic DNA | CP009182.1 |
| Mycobacterium tuberculosis | genomic DNA | CP009181.1 |
| Mycobacterium tuberculosis | genomic DNA | CP009180.1 |
| Mycobacterium tuberculosis | genomic DNA | CP009179.1 |
| Mycobacterium tuberculosis | genomic DNA | CP009178.1 |
| Mycobacterium tuberculosis | genomic DNA | CP009177.1 |
| Mycobacterium tuberculosis | genomic DNA | CP009176.1 |
| Mycobacterium tuberculosis | genomic DNA | CP009175.1 |
| Mycobacterium tuberculosis | genomic DNA | CP009174.1 |
| Mycobacterium tuberculosis | genomic DNA | CP009173.1 |
| Mycobacterium tuberculosis | genomic DNA | CP009172.1 |
| Mycobacterium tuberculosis | genomic DNA | CP009207.1 |
| Mycobacterium tuberculosis | genomic DNA | CP009204.1 |
| Mycobacterium tuberculosis | genomic DNA | CP009203.1 |

In one aspect, the gene that expresses transferrin is isolated from a mammal. In one aspect, the mammal species is a primate species such as, for example, a human, gorilla, bonobo, chimpanzee, gibbon, orangutan, macaque, baboon, lemur, Old World Monkey, or New World Monkey. In another aspect, the mammal species is a rodent species such as, for example, a ground squirrel, marmot, mouse, guinea pig, jerboa, chinchilla, degu, mole rat, or beaver. In still another aspect, the mammal species is a pika, tree shrew, or camel. In a further aspect, the gene that expresses transferrin has SEQ ID NO. 8 or at least 70% homology thereof, at least 75% homology thereof, at least 80% homology thereof, at least 85% homology thereof, at least 90% homology thereof, or at least 95% homology thereof. In one aspect, the gene that expresses transferrin is isolated from Homo sapiens and can be found in GenBank with GI number DQ923758.

Other sequences expressing transferrin or related or homologous genes can be identified in a database such as, for example, GenBank. In one aspect, sequences useful herein include those with the GI numbers listed in Table 5.

TABLE 5
Transferrin Genes

| | Sequence | |
|---|---|---|
| Source Organism | Description | GI Number |
| Homo sapiens | transferrin | NM_001063.3 |
| Homo sapiens | transferrin | DQ923758.1 |
| Gorilla gorilla | transferrin | XM_019022854.1 |
| | variant X1 | |
| Gorilla gorilla | transferrin | NM_001303546.1 |
| Homo sapiens | transferrin | S95936.1 |
| Homo sapiens | transferrin | XM_017007090.1 |
| | variant X2 | |
| Homo sapiens | transferrin | XM_017007089.1 |
| | variant X1 | |
| Homo sapiens | transferrin | M12530.1 |
| Homo sapiens | transferrin | AB590492.1 |
| Homo sapiens | transferrin | AK222755.1 |
| Homo sapiens | epididymis | GQ472199.1 |
| | secretory sperm | |
| | binding protein | |
| Pan paniscus | serotransferrin | XM_008976094.1 |
| Homo sapiens | transferrin | BC059367.1 |
| Homo sapiens | transferrin | KJ897654.1 |
| Homo sapiens | transferrin | CR936810.1 |
| Pan troglodytes | transferrin | NM_001144835.1 |
| Nomascus leucogenys | transferrin | XM_003265239.3 |
| Pongo pygmaeus | transferrin | KM972646.1 |
| Nomascus leucogenys | transferrin | NM_001308674.1 |
| Pongo abelii | transferrin | NM_001133958.1 |
| Homo sapiens | serotransferrin | AK295419.1 |
| | precursor | |
| Symphalangus syndactylus | transferrin | KM972647.1 |
| Hylobates lar | transferrin | KM972649.1 |
| Allenopithecus nigroviridis | transferrin | KM972653.1 |
| Homo sapiens | transferrin | BX648533.1 |
| Homo sapiens | transferrin | AF118063.1 |
| Trachypithecus francoisi | transferrin | KM972656.1 |
| Lophocebus albigena | transferrin | KM972652.1 |
| Colobus angolensis palliatus | serotransferrin | XM_011958201.1 |
| | variant X1 | |
| Chlorocebus sabaeus | transferrin | XM_008009084.1 |
| | variant X1 | |
| Macaca nemestrina | transferrin | XM_011721456.1 |
| Macaca fascicularis | transferrin | AB169522.1 |
| Colobus guereza | transferrin | KM972655.1 |
| Macaca fascicularis | serotransferrin | XM_005545793.2 |
| Cerocebus atys | transferrin | XM_012061466.1 |
| Mandrillus lecuophaeus | transferrin | XM_011970225.1 |
| | variant X2 | |
| Macaca mulatta | serotransferrin | NM_001318182.1 |
| Mandrillus leucophaeus | transferrin | XM_011970224.1 |
| | variant X1 | |
| Macaca fascicularis | transferrin | AB170458.1 |
| Pongo abelii | transferrin | XM_009239319.1 |
| Saimiri sciureus | transferrin | KM972659.1 |
| Saimiri boliviensis | transferrin | XM_003925066.2 |
| Aotus nancymaae | transferrin | NM_001308518.1 |
| Papio anubis | transferrin | KM972651.1 |
| Cercopithecus ascanius | transferrin | KM972654.1 |
| Chlorocebus sabaeus | transferrin | XM_008009085.1 |
| | variant X2 | |
| Papio anubis | serotransferrin | XM_003895098.3 |
| Rhinopithecus bieti | serotransferrin | XM_017872421.1 |
| | variant X2 | |
| Rhinopithecus bieti | serotransferrin | XM_017872420.1 |
| | variant X1 | |
| Lagothrix lagotricha | transferrin | KM972663.1 |
| Callithrix jacchus | transferrin | XM_008983803.2 |
| Callithrix geoffroyi | transferrin | KM972658.1 |
| Callicebus moloch | transferrin | KM972661.1 |
| Alouatta sara | transferrin | KM972662.1 |
| Homo sapiens | serotransferrin | AK295334.1 |
| | precursor | |
| Cebus capucinus imitator | transferrin | XM_017505719.1 |
| | variant X2 | |
| Homo sapiens | serotransferrin | AK303753.1 |
| | precursor | |
| Saguinus fuscicollis | transferrin | KM972657.1 |
| Cebus capucinus imitator | transferrin | XR_001818300.1 |
| | variant X1 | |
| Colobus angolensis | serotransferrin | XM_011958202.1 |
| | variant X2 | |
| Tarsius syrichta | serotransferrin-like | XM_008057562.1 |
| | protein | |
| Propithecus coquereli | serotransferrin-like | XM_012664058.1 |
| | protein | |
| Microcebus murinus | serotransferrin-like | XM_012766832.2 |
| | protein | |
| Galeopterus variegatus | serotransferrin-like | XM_008581124.1 |
| | protein | |
| Ochotona princeps | transferrin | XM_004588321.2 |
| | variant X1 | |
| Ictidomys tridecemileatus | serotransferrin | XM_005327026.1 |
| Oryctolagus cuniculus | transferrin | NM_001101694.1 |
| Marmota monax | transferrin | AY288100.1 |
| Ochotona princeps | transferrin | XM_004588320.2 |
| | variant X2 | |
| Marmota marmota | transferrin | XM_015487041.1 |
| Jaculus jaculus | transferrin | XM_004664199.1 |
| | variant X2 | |
| Chinchilla lanigera | serotransferrin | XM_005406809.2 |
| Nannospalax galili | serotransferrin-like | XM_017802840.1 |
| | protein | |
| Octodon degus | serotransferrin | XM_004625212.1 |
| Tupaia chinensis | transferrin | XM_014584459.1 |
| Heterocephalus glaber | serotransferrin | XM_004834254.3 |
| Cavia porcellus | serotransferrin | XM_003476728.3 |
| Fukomys damarensis | serotransferrin | XM_010625485.2 |
| | variant X2 | |
| Fukomys damarensis | serotransferrin | XM_010625484.2 |
| | variant X1 | |
| Peromyscus maniculatus bairdii | serotransferrin | XM_006978668.2 |
| Rhinopithecus roxellana | transferrin | XM_010379532.1 |
| Synthetic construct | fusion protein gene | JX091745.1 |
| Castor canadensis | serotransferrin-like | XM_020165722.1 |
| | protein | |
| Mus musculus | transferrin | NM_133977.2 |
| Mus musculus | transferrin | AK142599.1 |
| Mus musculus | transferrin | AK168419.1 |
| Mus musculus | transferrin | AK149559.1 |
| Mus musculus | transferrin | AK085754.1 |
| Camelus ferus | transferrin | XM_006179717.2 |
| Camelus dromedarius | transferrin | XM_010975530.1 |
| Camelus bactrianus | transferrin | XM_010947720.1 |
| Mus musculus | transferrin | AK149595.1 |
| Mus musculus | transferrin | BC022986.1 |
| Mus musculus | transferrin | BC012313.1 |
| Mus musculus | transferrin | BC092046.1 |
| Mus musculus | transferrin | BC058218.1 |
| Mus musculus | transferrin | BC058216.1 |
| Mus musculus | transferrin | BC020295.1 |
| Mus musculus | transferrin | AK168405.1 |
| Mus musculus | transferrin | AK150782.1 |

In one aspect, the gene that expresses flavonol synthase is isolated from a plant. In a further aspect, the gene that expresses flavonol synthase is isolated from a plant in the mustard family, or Brassicaceae. In one aspect, the mustard family species can be, for example, an Arabidopsis species (such as A. thaliana), pink shepherd's purse, saltwater cress, false flax, nakedstem wallflower, radish, wild cabbage (including any of the cultivars broccoli, cabbage, cauliflower, kale, Brussels sprouts, collard greens, or kohlrabi), turnip (including the Napa cabbage and bok choy cultivars), or canola (also known as rapeseed). In another aspect, the flavonol synthase is flavonol synthase 1. In a further aspect, the gene that expresses flavonol synthase has SEQ ID NO. 9 or at least 70% homology thereof, at least 75% homology thereof, at least 80% homology thereof, at least 85% homology thereof, at least 90% homology thereof, or at least 95% homology thereof. In one aspect, the gene that expresses flavonol synthase is isolated from Fragaria x ananassa (strawberry) and can be found in GenBank with GI number AAZ78661.1.

Other sequences expressing flavonol synthase or related or homologous genes can be identified in a database such as, for example, GenBank. In one aspect, sequences useful herein include those with the GI numbers listed in Table 6.

TABLE 6
Flavonol Synthase Genes

| Source Organism | Sequence Description | GI Number |
|---|---|---|
| Arabidopsis thaliana | flavonol synthase 1 | NM_120951.3 |
| Arabidopsis thaliana | flavonol synthase 1 | NM_001203337.1 |
| Arabidopsis thaliana | flavonol synthase | BT000494.1 |
| Arabidopsis thaliana | genomic DNA | AY058068.1 |
| Arabidopsis thaliana | flavonol synthase | U84259.1 |
| Arabidopsis thaliana | genomic DNA | AY086328.1 |
| Arabidopsis thaliana | flavonol synthase | U84260.1 |
| Arabidopsis lyrata | flavonol synthase/flavanone 3- | XM_021022274.1 |
| | hydroxylase | |
| Camelina sativa | flavonol synthase/flavanone 3- | XM_010424737.2 |
| | hydroxylase | |
| Camelina sativa | flavonol synthase/flavanone 3- | XM_010493211.2 |
| | hydroxylase-like protein | |
| Capsella rubella | hypothetical protein | XM_006288082.1 |
| Camelina sativa | flavonol synthase/flavanone 3- | XM_010454576.2 |
| | hydroxylase-like protein | |
| Eutrema salsugineum | hypothetical protein | XM_006399307.1 |
| Parrya nudicaulis | flavonol synthase | HQ215235.1 |
| Parrya nudicaulis | flavonol synthase | HQ215236.1 |
| Raphanus sativus | flavonol synthase/flavanone 3- | XM_018586825.1 |
| | hydroxylase | |
| Brassica oleracea | flavonol synthase/flavanone 3- | XM_013751217.1 |
| | hydroxylase | |
| Brassica rapa | flavonol synthase/flavanone 3- | XM_009124234.2 |
| | hydroxylase | |
| Brassica napus | flavonol synthase/flavanone 3- | XM_013860217.1 |
| | hydroxylase | |
| Arabidopsis thaliana | genomic DNA | CP002688.1 |
| Arabidopsis thaliana | flavone synthase, ecotype Col-2 | AM887647.1 |
| Arabidopsis thaliana | genomic DNA | AL590346.1 |
| Arabidopsis thaliana | flavonol synthase | U84258.1 |
| Arabidopsis thaliana | genomic DNA | AB006697.1 |
| Arabidopsis thaliana | flavonol synthase 1 | EU287459.1 |
| Arabidopsis thaliana | flavonol synthase G68R variant | EU287458.1 |
| Arabidopsis thaliana | flavone synthase, ecotype Ita-0 | AM887658.1 |
| Arabidopsis thaliana | flavone synthase, ecotype La-0 | AM887653.1 |
| Arabidopsis thaliana | flavone synthase, ecotype Tul-0 | AM887651.1 |
| Arabidopsis thaliana | flavone synthase, ecotype Gr-5 | AM887648.1 |
| Arabidopsis thaliana | flavone synthase, ecotype Mr-0 | AM887645.1 |
| Arabidopsis thaliana | flavone synthase, ecotype Rub-1 | AM887643.1 |
| Arabidopsis thaliana | flavone synthase, ecotype Cha-0 | AM887641.1 |
| Arabidopsis thaliana | flavone synthase, ecotype Ws-0 | AM887640.1 |
| Arabidopsis thaliana | flavone synthase, ecotype Kas-1 | AM887639.1 |
| Arabidopsis thaliana | flavonol synthase 1 | U72631.1 |
| Arabidopsis thaliana | flavone synthase, ecotype Can-0 | AM887657.1 |
| Arabidopsis thaliana | flavone synthase, ecotype Cvi-0 | AM887642.1 |
| Arabidopsis lyrata | flavone synthase | AM887659.1 |
| Arabis alpina | genomic DNA | LT669795.1 |
| Eutrema salsugineum | hypothetical protein | XM_006401952.1 |
| Parrya nudicaulis | flavonol synthase | HQ215393.1 |
| Parrya nudicaulis | flavonol synthase | HQ215392.1 |
| Parrya nudicaulis | flavonol synthase | HQ215387.1 |
| Parrya nudicaulis | flavonol synthase | HQ215406.1 |
| Parrya nudicaulis | flavonol synthase | HQ215405.1 |
| Parrya nudicaulis | flavonol synthase | HQ215402.1 |
| Parrya nudicaulis | flavonol synthase | HQ215401.1 |
| Parrya nudicaulis | flavonol synthase | HQ215400.1 |
| Parrya nudicaulis | flavonol synthase | HQ215399.1 |
| Parrya nudicaulis | flavonol synthase | HQ215398.1 |
| Parrya nudicaulis | flavonol synthase | HQ215397.1 |
| Parrya nudicaulis | flavonol synthase | HQ215396.1 |
| Parrya nudicaulis | flavonol synthase | HQ215395.1 |
| Parrya nudicaulis | flavonol synthase | HQ215394.1 |
| Parrya nudicaulis | flavonol synthase | HQ215391.1 |
| Parrya nudicaulis | flavonol synthase | HQ215385.1 |
| Parrya nudicaulis | flavonol synthase | HQ215384.1 |
| Parrya nudicaulis | flavonol synthase | HQ215408.1 |
| Parrya nudicaulis | flavonol synthase | HQ215409.1 |
| Parrya nudicaulis | flavonol synthase | HQ215403.1 |
| Parrya nudicaulis | flavonol synthase | HQ215388.1 |
| Arabidopsis lyrata | flavonol synthase 5 | XM_021022487.1 |
| Arabidopsis thaliana | flavonol synthase | EU287457.1 |

In one aspect, the gene that expresses iron oxidase is isolated from a yeast such as, for example, Komagataella phaffii (also known as Pichia pastoris). In another aspect, the gene that expresses iron oxidase is isolated from a bacterium of the Acidithiobacillus genus such as, for example, A. ferrivorans or ferrooxidans. In still another aspect, the gene that expresses iron oxidase is isolated from an insect such as a parasitic wasp or a fruit fly (e.g., Drosophila species D. virilis, D. serrata, D. miranda, D. pseudoobscura, D. willistoni, D. mojavensis, D. erecta, D. persimilis, D. rhopaloa, D. eugracilis, D. biarmipes, or D. grimshawi). In a different aspect, the gene that expresses iron oxidase is isolated from an Apicomplexan parasite such as, for example, Eimeria necatrix, E. mitis, or E. maxima. In still another aspect, the gene that expresses iron oxidase is isolated from a fungus. In a further aspect, the gene that expresses iron oxidase has SEQ ID NO. 10 or at least 70% homology thereof, at least 75% homology thereof, at least 80% homology thereof, at least 85% homology thereof, at least 90% homology thereof, or at least 95% homology thereof. In a still further aspect, the gene that expresses iron oxidase is a longer sequence that incorporates SEQ ID NO. 10. In one aspect, the gene that expresses iron oxidase can be isolated from Acidithiobacillus ferrivorans and can be found in GenBank with accession number AEM49324.1.

Other sequences expressing iron oxidase or related or homologous genes can be identified in a database such as, for example, GenBank. In one aspect, sequences useful herein include those with the GI numbers listed in Table 7.

TABLE 7
Iron Oxidase Genes

| Source Organism | Sequence Description | GI Number |
|---|---|---|
| Acidithiobacillus | genomic DNA | LT841305.1 |
| ferrivorans | | |
| Acidithiobacillus | genomic DNA | CP002985.1 |
| ferrivorans | | |
| Acidithiobacillus | high potential iron-sulfur | LC115034.1 |
| sp. NU-1 | protein | |
| Acidithiobacillus | high potential iron-sulfur | KC533886.1 |
| ferrivorans | protein | |
| Acidithiobacillus | genomic DNA | CP001219.1 |
| ferrooxidans | | |
| Acidithiobacillus | genomic DNA | CP001132.1 |
| ferrooxidans | | |
| Acidithiobacillus | putative cytochrome C1 | AJ320262.1 |
| ferrooxidans | and hip gene (high-redox | |
| | potential iron-sulfur | |
| | protein) | |
| Acidithiobacillus | extracellular iron oxidase | KP202695.1 |
| ferrooxidans | gene | |
| Acidithiobacillus | high potential iron-sulfur | AJ621387.2 |
| ferrooxidans | protein | |
| Acidithiobacillus | high potential iron-sulfur | AJ621388.2 |
| ferrooxidans | protein | |
| Acidithiobacillus | high potential iron-sulfur | AJ621389.1 |
| ferrooxidans | protein | |
| Acidithiobacillus | high potential iron-sulfur | FN688768.1 |
| ferrooxidans | protein | |
| Acidithiobacillus | high potential iron-sulfur | AJ621386.2 |
| ferrooxidans | protein | |
| Drosophila virilis | genomic DNA | XM_002051073.2 |
| Drosophila serrata | myc protein | XM_020952897.1 |
| Komagataella phaffii | genomic DNA | CP014716.1 |
| Komagataella phaffii | genomic DNA | CP014709.1 |
| Eimeria necatrix | hypothetical protein | XM_013579952.1 |
| Capsaspora owczarzaki | hypothetical protein | XM_004365711.2 |
| Komagataella phaffii | GS115 subunit of TFIID | XM_002491459.1 |
| | and SAGA complexes | |
| Pichia pastoris | genomic DNA | FN392320.1 |
| Mus pahari | zinc finger homeobox 3 | XM_021220667.1 |
| Amphimedon | serine/threonine-protein | XM_019994058.1 |
| queenslandica | phosphatase 6 | |
| Harpegnathos saltator | hypothetical protein | XM_019844601.1 |
| Aptenodytes forsteri | mediator complex subunit | XM_019474213.1 |
| | 12 | |
| Drosophila miranda | CREBRF homolog variant | XM_017287684.1 |
| | X2 | |
| Drosophila miranda | CREBRF homolog variant | XM_017287683.1 |
| | X1 | |
| Drosophila | uncharacterized protein | XM_003736903.2 |
| pseudoobscura | | |
| pseudoobscura | | |
| Drosophila | uncharacterized protein | XM_001360048.3 |
| pseudoobscura | | |
| pseudoobscura | | |
| Drosophila willistoni | uncharacterized protein | XM_002071081.2 |
| Drosophila mojavensis | uncharacterized protein | XM_002010833.2 |
| Drosophila erecta | uncharacterized protein | XM_001976758.2 |
| Trichogramma | basic salivary proline-rich | XM_014377751.1 |
| pretiosum | protein 1-like variant X2 | |
| Trichogramma | basic salivary proline-rich | XM_014377750.1 |
| pretiosum | protein 1-like variant X1 | |
| Eimeria mitis | hypothetical protein | XM_013498177.1 |
| Eimeria mitis | hypothetical protein | XM_013497016.1 |
| Eimeria mitis | hypothetical protein | XM_013496741.1 |
| Eimeria mitis | hypothetical protein | XM_013495477.1 |
| Eimeria mitis | hypothetical protein | XM_013494226.1 |
| Eimeria maxima | hypothetical protein | XM_013477429.1 |
| Sordaria macrospora | k-hell hypothetical protein | XM_003345950.1 |
| Drosophila persimilis | uncharacterized protein | XM_002019945.1 |
| Grapholita molesta | microsatellite sequence | KX711552.1 |
| Drosophila rhopaloa | BEACH domain- | XM_017123494.1 |
| | containing protein IvsC, | |
| | variant X5 | |
| Drosophila rhopaloa | BEACH domain- | XM_017123493.1 |
| | containing protein IvsC, | |
| | variant X4 | |
| Drosophila rhopaloa | BEACH domain- | XM_017123492.1 |
| | containing protein IvsC, | |
| | variant X3 | |
| Drosophila rhopaloa | BEACH domain- | XM_017123491.1 |
| | containing protein IvsC, | |
| | variant X2 | |
| Drosophila rhopaloa | BEACH domain- | XM_017123490.1 |
| | containing protein IvsC, | |
| | variant X1 | |
| Drosophila eugracilis | insulin-like receptor | XM_017208806.1 |
| Drosophila biarmipes | putative uncharacterized | XM_017091657.1 |
| | protein | |
| Acyrthosiphon pisum | Krueppel-like factor 6 | XM_003240097.3 |
| Neodiprion lecontei | uncharacterized protein | XM_015666154.1 |
| Trichogramma | putative uncharacterized | XM_014364909.1 |
| pretiosum | protein | |
| Eimeria necatrix | hypothetical protein | XM_013582418.1 |
| Eimeria mitis | hypothetical protein | XM_013497930.1 |
| Eimeria mitis | hypothetical protein | XM_013496691.1 |
| Eimeria mitis | hypothetical protein | XM_013496040.1 |
| Eimeria maxima | hypothetical protein | XM_013478862.1 |
| Enterobius vermicularis | genomic DNA | LM416156.1 |
| Drosophila persimilis | uncharacterized protein | XM_002024972.1 |
| Drosophila grimshawi | uncharacterized protein | XM_001990136.1 |

In one aspect, the gene that expresses zinc oxidase is isolated from bacteria. In a further aspect, the bacteria are Streptomyces bacteria such as, for example, S. lincolnensis, S. collinus, S. avermitilis, S. parvulus, S. ambofaciens, S. scabiei, S. davawensis, S. pluripotens, S. pactum, S. puniciscabiei, S. griseochromogenes, S. incarnatus, S. aureocfaciens, S. reticuli, S. hygroscopicus, S. fulvissimus, S. katrae, S. silaceus, S. venezuelae, or S. albireticuli. In another aspect, the bacteria are Clostridium bacteria such as C. sporogenes or C. botulinum. In still another aspect, the bacteria are selected from one of the following genera: Polaribacter, Kitasatospora, Actinobacteria, Azospirillum, Collimonas, or Micromonospora. In a different aspect, the gene that expresses zinc oxidase is isolated from algae. In one aspect, the algal species is, for example, Guillardia theta. In an alternative aspect, the gene that expresses zinc oxidase is isolated from fish. In one aspect, the fish is salmon. In a further aspect, the gene that expresses zinc oxidase has SEQ ID NO. 11or at least 70% homology thereof, at least 75% homology thereof, at least 80% homology thereof, at least 85% homology thereof, at least 90% homology thereof, or at least 95% homology thereof. In a still further aspect, the gene that expresses zinc oxidase is a longer sequence that incorporates SEQ ID NO. 11. In one aspect, the gene that expresses zinc oxidase is isolated from Streptomyces zinciresistens and can be found in GenBank with GI number EGX59011.1.

Other sequences expressing zinc oxidase or related or homologous genes can be identified in a database such as, for example, GenBank. In one aspect, sequences useful herein include those with the GI numbers listed in Table 8.

TABLE 8
Zinc Oxidase Genes

| | Sequence | |
|---|---|---|
| Source Organism | Description | GI Number |
| Streptomyces lincolnensis | genomic DNA | CP016438.1 |
| Streptomyces sp. 4F | genomic DNA | CP013142.1 |
| Streptomyces collinus | genomic DNA | CP006259.1 |
| Streptomyces avermitilis | genomic DNA | BA000030.4 |
| Streptomyces sp. 3124.6 | genomic DNA | LT670819.1 |
| Streptomyces parvulus | genomic DNA | CP015866.1 |
| Streptomyces ambofaciens | genomic DNA | CP012949.1 |
| Streptomyces ambofaciens | genomic DNA | CP012382.1 |
| Streptomyces scabiei | genomic DNA | FN554889.1 |
| Streptomyces davawensis | genomic DNA | HE971709.1 |
| Polaribacter sp. SA4-12 | genomic DNA | CP019334.1 |
| Streptomyces sp. CdTB01 | genomic DNA | CP013743.1 |
| Kitasatospora setae | genomic DNA | AP010968.1 |
| Streptomyces pluripotens | genomic DNA | CP021080.1 |
| Streptomyces pactum | genomic DNA | CP019724.1 |
| Polaribacter sp. Hel1 | genomic DNA | LT629794.1 |
| Streptomyces sp. TLI | genomic DNA | LT629775.1 |
| Streptomyces pactum | genomic DNA | CP016795.1 |
| Streptomyces puniciscabiei | genomic DNA | CP017248.1 |
| Streptomyces griseochromogenes | genomic DNA | CP016279.1 |
| Streptomyces incarnatus | genomic DNA | CP011497.1 |
| Streptomyces aureofaciens | genomic DNA | CP020567.1 |
| Streptomyces sp. S10(2016) | genomic DNA | CP015098.1 |
| Streptomyces reticuli | genomic DNA | LN997842.1 |
| Actinobacteria bacterium IMCC25003 | genomic DNA | CP015603.1 |
| Polaribacter sp. KT25b | genomic DNA | LT629752.1 |
| Streptomyces hygroscopicus subsp. | genomic DNA | CP013219.1 |
| limoneus | | |
| Streptomyces sp. Mg1 | genomic DNA | CP011664.1 |
| Azospirillum brasiliense | genomic DNA | CP007796.1 |
| Streptomyces hygroscopicus subsp. | genomic DNA | CP003720.1 |
| jinggangensis | | |
| Streptomyces hygroscopicus subsp. | genomic DNA | CP003275.1 |
| jinggangensis | | |
| Collimonus arenae | genomic DNA | CP009962.1 |
| Polaribacter sp. MED152 | genomic DNA | CP004349.1 |
| Streptomyces sp. S8 | genomic DNA | CP015362.1 |
| Micromonospora echinofusca | genomic DNA | LT607733.1 |
| Streptomyces sp. PBG53 | genomic DNA | CP011799.1 |
| Streptomyces fulvissimus | genomic DNA | CP005080.1 |
| Streptomyces katrae | genomic DNA | CP020042.1 |
| Streptomyces silaceus | genomic DNA | CP015588.1 |
| Streptomyces venezuelae | genomic DNA | CP018074.1 |
| Salmo solar | calmodulin | XM_014213459.1 |
| Streptomyces venezuelae | genomic DNA | FR845719.1 |
| Salmo solar | calmodulin | BT059493.1 |
| Salmo solar | calmodulin | BT045544.1 |
| Streptomyces albireticuli | genomic DNA | CP021744.1 |
| Streptomyces sp. 3211 | genomic DNA | CP020039.1 |
| Clostridium sporogenes | genomic DNA | CP011663.1 |
| Clostridium sporogenes | genomic DNA | CP009225.1 |
| Clostridium botulinum | genomic DNA | CP006902.1 |
| Guillardia theta | hypothetical | XM_005830304.1 |
| | protein | |

In one aspect, the DNA constructs disclosed herein include a reporter protein. In a further aspect, the reporter protein is a yellow fluorescent reporter protein. In a still further aspect, the gene that expresses the yellow fluorescent reporter protein has SEQ ID NO. 12 or at least 70% homology thereof, at least 75% homology thereof, at least 80% homology thereof, at least 85% homology thereof, at least 90% homology thereof, or at least 95% homology thereof.

Exemplary methods for producing the DNA constructs described herein are provided in the Examples. Restriction enzymes and purification techniques known in the art can be used to prepare the DNA constructs. After the vector incorporating the DNA construct has been produced, it can be incorporated into host cells using the methods described below.

### Biological Devices

In one aspect, a “biological device” is formed when a microbial cell is transfected with the DNA construct described herein. The biological devices are generally composed of microbial host cells, where the host cells are transformed with a DNA construct described herein.

In one aspect, the DNA construct is carried by the expression vector into the cell and is separate from the host cell's genome. In another aspect, the DNA construct is incorporated into the host cell's genome. In still another aspect, incorporation of the DNA construct into the host cell enables the host cell to produce

UV-protective proteins.

The host cells as referred to herein include their progeny, which are any and all subsequent generations formed by cell division. It is understood that not all progeny may be identical due to deliberate or inadvertent mutations. A host cell may be “transfected” or “transformed,” which refers to a process by which exogenous nucleic acid is transferred or introduced into the host cell. A transformed cell includes the primary subject cell and its progeny. The host cells can be naturally-occurring cells or “recombinant” cells. Recombinant cells are distinguishable from naturally-occurring cells in that naturally-occurring cells do not contain heterologous nucleic acid sequences introduced using molecular biology techniques. In one aspect, the host cell is a prokaryotic cell such as, for example, Escherichia coli. In other aspects, the host cell is a yeast such as, for example, Saccharomyces cerevisiae. Host cells transformed with the DNA construct described herein are referred to as biological devices.

The DNA construct is first delivered into the host cell. This delivery can be accomplished in vitro, using well-developed laboratory procedures for transforming cell lines. Transformation of bacterial cell lines can be achieved using a variety of techniques. One method involves calcium chloride. The exposure to the calcium ions renders the cells able to take up the DNA construct. Another method is electroporation. In this technique, a high-voltage electric field is applied briefly to cells, producing transient holes in the cell membrane through which the vector containing the DNA construct enters. Exemplary procedures for transforming yeast and bacteria with specific DNA are provided in the Examples. In certain aspects, two or more types of DNA can be incorporated into the host cells. Thus, different metabolites can be produced from the same plant at enhanced rates.

Once the DNA construct has been incorporated into the host cell, the cells are cultured such that the cells multiply. A satisfactory microbiological culture contains available sources of hydrogen donors and acceptors, carbon, nitrogen, sulfur, phosphorus, inorganic salts, and, in certain cases, vitamins or other growth promoting substances. The addition of peptone provides a readily available source of nitrogen and carbon. A variety of other carbon sources are contemplated, including, but not limited to: monosaccharides such as glucose and fructose, disaccharides such as lactose and sucrose, oligosaccharides, polysaccharides such as starch, and mixtures thereof. Unpurified mixtures extracted from feedstocks are also contemplated and can include molasses, barley malt, and related compounds and compositions. Other glycolytic and tricarboxylic acid cycle intermediates are also contemplated as carbon sources, as are one-carbon substrates such as carbon dioxide and/or methanol in the cases of compatible organisms. The carbon source utilized is limited only by the particular organism being cultured.

Furthermore, the use of different media results in different growth rates and different stationary phase densities. Secondary metabolite production is highest when cells are in stationary phase. A rich media results in a short doubling time and higher cell density at stationary phase. Minimal media results in slow growth and low final cell densities. Efficient agitation and aeration increase final cell densities. A skilled artisan will be able to determine which type of media is best suited to culture a particular species and/or strain of host cell.

Culturing or fermenting host cells may be accomplished by any technique known in the art. In one aspect, batch fermentation can be conducted. In batch fermentation, the composition of the culture medium is set at the beginning of culturing and the system is closed to future artificial alterations. In some aspects, a limited form of batch fermentation can be carried out, wherein factors such as oxygen concentration and pH are manipulated, but additional carbon is not added. Continuous fermentation methods are also contemplated. In continuous fermentation, equal amounts of a defined medium are continuously added to and removed from a bioreactor. In other aspects, microbial host cells are immobilized on a substrate. Fermentation can be carried out on any scale and may include methods in which literal “fermentation” is carried out as well as other culture methods that are non-fermentative.

### UV-Resistant Microbes

In one aspect, a yeast or bacterium, particularly a beneficial yeast or bacterium such as one used in a fermentation process, can be engineered to be UV-resistant by transforming or transfecting the yeast or bacteria with a nucleic acid able to express a protein up-regulated by UV exposure. In one aspect, the nucleic acids can be under the control of a constitutive promoter. In another aspect, the nucleic acids can be under control of a UV-inducible promoter. In some aspects, when the yeast or bacterium needs to perform another function, such as fermentation, the nucleic acids can be under control of UV-inducible promoters so as not to impede the other function when UV protection is not required.

In a further aspect, UV-resistant microbes can be used in fermentation processes, such as the production of alcohol or fuel ethanol, or in the production of chemical and pharmaceutical products, including biological drug products.

### Preparation of UV-Protective Extracts

In one aspect, provided herein are UV-protective extracts produced by the biological devices disclosed herein. In one aspect, the present UV-protective microbial extracts are able to wholly or partially block the passage of UV radiation. The extent to which UV radiation is blocked can depend on a variety of factors including the particular microbe used, the amount of extract applied, and the formulation of the extract.

In a further aspect, the UV protective extract can be prepared by exposing a culture of a biological device such as those disclosed herein to UV radiation, then extracting components from the culture. In one aspect, the components are extracted via centrifugation. In another aspect, the culture of the biological device is applied to a subject or surface after UV irradiation and without extraction. The UV radiation can be of any wavelength, but in one aspect, it can be shortwave radiation (i.e., ultraviolet C having a wavelength of approximately 100 to 280 nm), medium wave radiation (i.e., ultraviolet B, having a wavelength of approximately 280 to 315 nm), or longwave radiation (i.e., ultraviolet A having a wavelength of 315 to 400 nm). In one aspect, the culture of the biological device can be irradiated with a 254 nm shortwave UV source. In another aspect, the culture of the biological device can be irradiated with a 365 nm longwave UV source. In still another aspect, the culture of the biological device can be irradiated with both a 254 nm and a 365 nm UV source. In yet another aspect, the culture of the biological device can be irradiated with a natural

UV source such as, for example, the sun, providing a range of wavelengths for irradiation.

In one aspect, culture of the biological device may proceed until the culture is dense, but not so dense as to trigger deleterious responses (e.g., a response triggered by lack of a food source) and not so dense as to prevent UV radiation from reaching a substantial portion of cells in the culture. Once the desired culture density has been reached, the culture can then be irradiated with UV radiation. Prior to irradiation, in one aspect, the culture is transferred to one or more vessels designed to allow a substantial portion of the biological device to be irradiated.

In one aspect, the irradiation continues for the length of time needed to induce a radiation response in the biological devices and ends at or before a time at which a substantial portion of the biological devices are fatally irradiated. In a further aspect, the extract can be collected after exposing a culture of a biological device to UV irradiation for a period of time ranging from about 12 hours to about 72 hours, or about 12, 24, 36, 48, 60, or 72 hours. In an alternative aspect, the biological devices may continue to be cultured for a time after UV exposure at least sufficient to allow some radiation response in the biological devices. In a further aspect, if irradiation did not cause death of a substantial portion of the organisms in culture, culture may continue until the radiation response has ceased in a majority of the organisms.

In one aspect, radiation response can include upregulation of at least one of the following: a hexokinase, a heat shock protein, an alcohol dehydrogenase, transferrin, a flavonol synthase, a zinc oxidase, an iron oxidase, or a combination thereof.

It will be understood that up-regulation or down-regulation of one or more of these proteins may not be directly responsible for UV-protective properties, such that increased or decreased amounts of these proteins in the extract may have little or no effect on the UV-protective properties of the extract. Further in this aspect, up-regulation or down-regulation of one of these proteins may have downstream effects that ultimately produce a UV-protective effect. In an alternative aspect, up-regulation or down-regulation of one or more of these proteins may be directly responsible for the UV-protective properties of the extract.

In one aspect, the extract is prepared in a manner able to isolate at least one UV-protective component. In some aspects, the extract can include centrifuged bacterial or yeast components. In one aspect, the extract is formulated at a variety of concentrations in any acceptable carrier to allow its use for a particular purpose. In some aspects, the extract is formulated in an evaporable carrier, such as water or alcohol, to allow the extract to dry on the surface of the material to be protected from UV radiation. In an alternative aspect, the extract is formulated in a lotion, gel, oil, or cream for application to human or animal skin.

In one aspect, the extract can be prepared by centrifuging the culture of biological devices in a manner able to precipitate most proteins, including UV-resistant and/or UV-protective proteins, then discarding the supernatant while retaining the pellet as the extract. Further in this aspect, the pellet can be used as-is or dried. Still further in this aspect, the pelleted material can be diluted to a given concentration using any acceptable carrier, such as water, alcohol, lotion, gel, oil, or cream. In one aspect, the carrier is non-denaturing. In an alternative aspect, the carrier is denaturing. In a still further aspect, the carrier also includes materials to inhibit further bacterial growth and/or protein degradation.

In an alternative aspect, the supernatant contains UV-protective compounds and is not discarded. In yet another aspect, the UV-protective and/or UV-resistant compounds and proteins are extracted by another method known in the art for isolating proteins and/or metabolites.

In a further aspect, the biological device culture may not be pelletized but instead may be killed, for example by lysis or exposure to lethal levels of UV radiation, and the culture medium can be used as-is or in an evaporated form. Further in this aspect, materials to inhibit further microorganism growth and/or protein degradation can also be introduced.

In another aspect, cells from any of the cultures described above can be isolated with or without extraction and/or lysis and used in wet or dry form.

In another aspect, isolated proteins from the biological device culture can be used in place of a more general extract to produce a UV-protective effect. Such proteins can be isolated by techniques known in the art.

### Applications of UV-Protective Extracts

The extract may be applied to any material that may benefit from a reduction in UV radiation. The exact formulation of the extract plus any carriers can be adjusted based on the desired use. In one aspect, the extract is formulated with only non-toxic components if it is to be used on a human or animal or with another microorganism, such as in a fermentation process or on an agricultural product. In another aspect, the extract can be mixed with other substances to provide UV-protective properties to the overall composition. In still another aspect, if coated on the material to be protected, the extract itself can be covered with a further protective coating to project, for example, against mechanical wear and damage.

In the case when the extract is applied to the surface of an article, it can be applied using techniques known in the art such spraying or coating. In other aspects, the extract can be intimately mixed with a substance or material that ultimately produces the article. For example, the extract can be mixed with molten glass so that the extract is dispersed throughout the final glass product.

In one aspect, the extract is formulated or applied in such a manner as to block approximately 50%, 60%, 70%, 80%, 90%, 95%, or 99% of the UV radiation that encounters the extract, where any value can be a lower and upper end-point of a range (e.g., 60% to 95%). In a further aspect, the extract can also be formulated to block these percentages of particular UV wavelengths, or, more generally, to block these percentages of UVA, UVB, or UVC radiation.

Extracts according to the present disclosure can be used for a variety of purposes. These purposes include, but are not limited to, the following:

1. blocking UV radiation or other types of radiation;
2. protecting human skin against damage and/or skin cancer induced by UV radiation or other types of radiation;
3. protecting against side effects of radiation used in cancer treatments;
4. protecting animals from deleterious effects of UV radiation or other radiation;
5. protecting plastic, fiberglass, glass, rubber, or other solid surfaces from UV radiation or other radiation;
6. providing a UV radiation screen or screen for other types of radiation;
7. protecting astronauts and/or other persons or organisms as well as equipment during space trips;
8. enhancement of industrial fermentation processes or other processes requiring energy by allowing the use of UV radiation in connection with the process to supply additional energy and thus to increase the ultimate energy-requiring output of the cells without substantially killing the fermenting organism;
9. protection of experimentation, fermentation, biochemical, and/or biological processes under the presence of UV radiation, for example in extraterrestrial conditions such as on the moon or Mars; and
10. protection of agricultural plants, particularly agricultural plants in which the revenue-producing part of the plant is above ground, such as fruits, vine vegetables, beans and peas, and leaf vegetables.

In one particular embodiment, an extract prepared according to the procedure described above, can be applied to an agricultural plant. In one aspect, the plant can be one that produces fruit or vegetable, such as, for example, a watermelon or a tomato. Further in this aspect, the extract can be applied during at least a part of the plant's growth to increase the amounts of one or more nutrients of the fruit or vegetable, such as a vitamin, mineral, or other recommended dietary component. In one specific aspect, the amount of lycopene can be increased (which may be accompanied by a decrease in carotene or other less-valuable nutrients formed by competing pathways). In another aspect, the amount of a flavor-enhancing component, such as glucose, can be increased. Further in this aspect, an increase in glucose can help protect against water loss.

In one aspect, the extract can be applied for about 25%, 50%, 75%, 90%, 95%, or 99% of the fruit or vegetable's on-plant life, where the on-plant life includes the time span from the formation of a separate body that will constitute the fruit or vegetable (in some aspects, excepting flowers) until the fruit or vegetable is harvested. In one aspect, the extract can be first applied when the fruit or vegetable is sufficiently large to no longer be substantially protected from UV radiation by leaves. In another aspect, the extract can first be applied five days, one week, or two weeks prior to harvest. Further in this aspect, application at this later stage can be particularly useful with fruits or vegetables in which an increase in a nutrient or flavor-enhancing component can be obtained by protecting the fruit or vegetable from UV radiation later in its on-plant life.

In one aspect, the extract can be applied once or multiple times to each fruit or vegetable. In another aspect, it can be applied weekly, or it can be reapplied after the fruit or vegetable is exposed to rain or after a turning process. In another aspect, the agricultural plant can be another food crop that grows above ground and is exposed to natural UV radiation, wherein the agricultural product produced can be a fruit, leaf, seed, flower, grain, nut, stem, vegetable, or mushroom.

In another aspect, it is desirable for agricultural plants that do not produce parts typically consumed by humans to be protected from UV irradiation. In a further aspect, these other agricultural plants can includes sources of fibers such as, for example, cotton and linen (flax), of cork, of wood or lumber, of feedstocks for producing ethanol or biodiesel (including, but not limited to, sugar beet, sugarcane, cassava, sorghum, corn, wheat, oil palm, coconut, rapeseed, peanut, sunflower, soybean, and the like), of animal feedstocks or fodder, or of decorative or horticultural plants.

In one aspect, any part of the plant can be coated, including, but not limited to, the part of the plant that is collected during harvest. In an alternative aspect, the harvested part of the plant is not coated, but another part can be coated with the extracts disclosed herein. In addition to the aspects already described, in one aspect, coating a plant with the extracts described herein can prolong the life of the plant, increase production capacity of a desired product, can increase the growth rate of the plant relative to an untreated plant of the same type, can increase production of a desired metabolite that might otherwise decrease due to UV-induced stress, can increase yield of a crop of such plants, and the like.

In a further aspect, application can be accomplished with a commercial sprayer. In another aspect, application can be only on the upper portions of the fruit or vegetable, which are exposed to substantially greater amounts of UV radiation than the lower portions of the fruit or vegetable.

In another aspect, provided herein is a pharmaceutical composition containing the extracts produced by the biological devices described herein. In one aspect, the pharmaceutical composition can be applied to a subject, wherein the subject is exposed to radiation. In one aspect, the radiation is applied as a strategy to treat cancer. In another aspect, the pharmaceutical composition is used to prevent radiation-induced cellular and DNA damage. In another aspect, dosage ranges of the extract in the pharmaceutical composition can vary from 0.01 g extract/mL of pharmaceutical composition to 1 g extract/mL of pharmaceutical composition, or can be 0.01, 0.02, 0.025, 0.05, 0.075, or 1 g extract/mL of pharmaceutical composition. In an alternative aspect, provided herein is a cosmetic composition containing the extracts produced by the biological devices described herein. Further in this aspect, the cosmetic composition can be a cleanser, lotion, cream, shampoo, hair treatment, makeup, lip treatment, nail treatment, or related composition. In still a further aspect, the compositions containing the extracts can have both pharmaceutical and cosmetic applications. In yet another aspect, the compositions containing the extracts can be used in veterinary medicine.

The cosmetic compositions can be formulated in any physiologically acceptable medium typically used to formulate topical compositions. The cosmetic compositions can be in any galenic form conventionally used for a topical application such as, for example, in the form of dispersions of aqueous gel or lotion type, emulsions of liquid or semi-liquid consistency of the milk type, obtained by dispersing a fatty phase in an aqueous phase (O/W) or vice versa (W/O), or suspensions or emulsions of soft, semi-solid or solid consistency of the cream or gel type, or alternatively multiple emulsions (W/O/VV or O/W/O), microemulsions, vesicular dispersions of ionic and/or non-ionic type, or wax/aqueous phase dispersions. These compositions are prepared according to the usual methods.

The cosmetic compositions can also contain one or more additives commonly used in the cosmetics field, such as emulsifiers, preservatives, sequestering agents, fragrances, thickeners, oils, waxes or film-forming polymers. In one aspect, in any of the above scenarios, the pharmaceutical, cosmetic, or veterinary composition also includes additional UV-protective compounds or UV-blocking agents such as, for example, zinc oxide, titanium dioxide, carotenoids, oxybenzone, octinoxate, homosalate, octisalate, octocrylene, avobenzone, or a combination thereof.

In one aspect, the composition is a sunscreen. A sunscreen can be formulated with any of the extracts produced herein. In addition to the extract, the sunscreen in certain aspects can be formulated with one or more UV-protective compounds or UV-blocking agents listed above. The sunscreen can be formulated as a paste, lotion, cream, aerosol, or other suitable formulations for topical use. In certain aspects, the sunscreen can be formulated as a transparent composition.

In one aspect, the cosmetic composition can be a film composed of the extracts produced herein that can be directly applied to the skin. For example, the film can be composed of a biocompatible material such as a protein or oligonucleotide, where the extract is coated on one or more surfaces of the film or, in the alternative dispersed throughout the film. For example, the film can be composed of DNA. In this application, the films can be used as a wound covering and provide protection from UV photodamage. The films can also be prepared so that they are optically transparent. Here, it is possible to view the wound without removing the covering and exposing the wound. The films can also include other components useful in cosmetic applications such as, for example, compounds to prevent or reduce wrinkles.

In one aspect, the pharmaceutical, cosmetic, or veterinary compositions described herein are applied to subjects. In one aspect, the subject is a human, another mammal, or a bird. In a further aspect, the mammal is a pet such as a dog or cat or is livestock such as horses, goats, cattle, sheep, and the like. In an alternative aspect, the bird is a pet bird or is poultry such as, for example, a chicken or turkey. In any of these aspects, the compositions can be applied to skin, fur, feathers, wool, hooves, horns, or hair as appropriate and applicable.

In another aspect, provided herein is a paint, dye, stain, or ink containing the UV-protective and/or UV-resistant extract disclosed herein. In one aspect, there are several benefits to having a paint that is resistant to UV irradiation. In a further aspect, imparting UV resistance to a paint slows or stops photodegradation, bleaching, or color fading. In another aspect, a paint with UV resistance prevents chemical modification of exposed paint surfaces. Further in this aspect, chemical modification of exposed paint surfaces includes change in finish, structural changes in binders, flaking, chipping, and the like. In one aspect, the paint provided herein resists these changes.

In still another aspect, provided herein is an article coated with the extracts disclosed herein. In one aspect, the article is made of glass, plastic, metal, wood, fabric, or any combination thereof. In one aspect, the article is a construction material such as, for example, steel, concrete or cement, brick, wood, window or door glass, fiberglass, siding, wallboard, a flooring material, masonry, mortar, grout, stone, artificial stone, stucco, shingles, roofing materials, and the like. In an alternative aspect, the material is an aeronautical or aerospace material such as, for example, the metal or metal alloy body of an aircraft or spacecraft, paint on the body of an aircraft or spacecraft, glass windows on an aircraft or spacecraft, carbon fiber composite, titanium or aluminum, a ceramic heat absorbing tile, and the like. In still another aspect, the article is a fabric article such as, for example, clothing, drapes, outdoor upholstery, a tent or outdoor pavilion, a flag or banner, or the like. In another aspect, the extract can be applied to the article to fine artwork, solid pieces (e.g., vases), and historical documents in order to preserve them. In another aspect, the extract can be applied to outdoor signs such as highway billboards and advertising.

In other aspects, the extract can be incorporated within or throughout the article. In one aspect, the extract can be mixed with molten glass to produce glass article that are UV resistant such as, for example, sunglasses, car windshields, window glass, and eyeglasses. In another aspect, the glass article can be a bottle for storing a beverage or food container in order to increase the shelf-life of the beverage or food. It is contemplated that the extract can be applied externally to the glass articles as well.

In another aspect, the extract can be mixed with fiberglass or plastics in order to reduce negative effects to aircraft, watercraft, boats, jet skis, decking, house siding, motor homes, sunroofs, and moon roofs that are constantly exposed to UV radiation. It is contemplated that the extract can be applied externally to the fiberglass or plastic articles as well.

In another aspect, the extract can be mixed with rubber, silicon, or latex used to make a variety of articles such as water hoses, tires, and the like. It is contemplated that the extract can be applied externally to the rubber, silicone, or latex articles as well.

In another aspect, the extract can be mixed with foams used to make a variety of articles such as automotive dashboard padding, seat cushions, and the like. It is contemplated that the extract can be applied externally to the foam articles as well.

In another aspect, the extracts described herein can be incorporated into an optical film. In one aspect, the extract is applied to at least one surface of the film. In another aspect, the extract can be dispersed throughout the film. The film can be transparent, translucent or opaque. The film can be composed of, but not limited to, polyolefin resin, such as polyethylene (PE) or polypropylene (PP); polyester resin, such as polyethylene terephthalate (PET); polyacrylate resin, such as polymethyl (meth) acrylate (PMMA); polycarbonate resin; polyurethane resin or a mixture thereof.

The optical film can be applied to any substrate where it is desirable to reduce or prevent UV exposure or damage. For example, the optical film can be applied to windows to reduce or prevent UV radiation from entering a structure (e.g., building, vehicle, etc.).

In another aspect, provided herein is a method of reducing or preventing the exposure of an item to UV radiation by applying the extracts described herein to the item or incorporating the extract within/throughout the article. Further in this aspect, “reducing” is defined relative to an untreated control. That is, if two like items are exposed to equal amounts of UV radiation for an equal amount of time, but one has been treated with the UV-resistant extracts and the other has not, and some objective response is measured (e.g., color fading, structural degradation, plant size or yield, etc.), the treated item will appear to have been exposed to a lower amount of UV (for example, the color of the treated item will have faded less and will remain closer to the original, or a treated plant will appear larger and more vigorous and will have a greater yield, etc.). In some aspects, treatment with the extracts disclosed herein will prevent UV exposure from occurring. As used herein, “prevent” indicates that a treated item will not be affected, changed, or altered by UV exposure.

In one aspect, the extract blocks from 50% to 100% of UV radiation from contacting the item. Further in this aspect, the extract blocks at least 50%, 55%, 60%, 65%, 70%, 75%, 80%, 85%, 90%, 95%, 99%, or 100% of UV radiation from contacting the item. In another aspect, the extract blocks from 50% to 100% of longwave UV radiation from contacting the item. Further in this aspect, the extract blocks at least 50%, 55%, 60%, 65%, 70%, 75%, 80%, 85%, 90%, 95%, 99%, or 100% of longwave UV radiation from contacting the item. In one aspect, the extract blocks from 50% to 100% of shortwave UV radiation from contacting the item. Further in this aspect, the extract blocks at least 50%, 55%, 60%, 65%, 70%, 75%, 80%, 85%, 90%, 95%, 99%, or 100% of shortwave UV radiation from contacting the item.

Depending upon the application, the extract can prevent or reduce damage cause by UV radiation from limited to extended periods of time. By varying the amount of extract that is applied as well as the number of times the extract is applied, the degree of UV protection can be varied. In certain aspects, it may be desirable for the article to be protected from UV damage for a short period of time then subsequently biodegrade.

In another aspect, the extracts produced herein can be used to reduce or prevent the growth of barnacles on boats and other water vehicles. In one aspect, the extract can be admixed with a paint that is typically applied to water vehicles, where the paint also includes chitosan. In one aspect, the chitosan can be acetylated to a specific degree of acetylation in order to enhance tissue growth during culturing as well as metabolite production. In one aspect, the chitosan is from 60% to about 100%, 70% to 90%, 75% to 85%, or about 80% acetylated. In one aspect, chitosan isolated from the shells of crab, shrimp, lobster, and/or krill is useful herein. The molecular weight of the chitosan can vary, as well. For example, the chitosan comprises about 3, 4, 5, 6, 7, 8, 9, 10, 11, 12, 13, 14, 15, 16, 17, 18, 19, or 20 glucosamine units and/or N-acetylglucosamine units. In another aspect, the chitosan includes 5 to 7 glucosamine units and/or N-acetylglucosamine units.

### UV-Resistant Plants

In one aspect, provided herein is a plant that is resistant to UV radiation. As used herein, “plant” is used in a broad sense to include, for example, any species of woody, ornamental, crop, cereal, fruit, or vegetable plant, as well as photosynthetic green algae. “Plant” also refers to a plurality of plant cells that are differentiated into a structure that is present at any stage of a plant's development. Such structures include, but are not limited to, fruits, shoots, stems, leaves, flower petals, roots, tubers, corms, bulbs, seeds, gametes, cotyledons, hypocotyls, radicles, embryos, gametophytes, tumors, and the like. “Plant cell,” “plant cells,” or “plant tissue” as used herein refers to differentiated and undifferentiated tissues of plants including those present in any of the tissues described above, as well as to cells in culture such as, for example, single cells, protoplasts, embryos, calluses, etc. It is contemplated that any cell from which a fertile plant can be regenerated is useful as a recipient cell. Type I, Type II, and Type III callus can be initiated from tissue sources including, but not limited to, immature embryos, immature inflorescences, seedling apical meristems, microspores, and the like. Those cells that are capable of proliferating as callus are also useful herein. Methods for growing plant cells are known in the art (see U.S. Pat. No. 7,919,679).

In one aspect, plant calluses grown from 2 to 4 weeks can be used herein. The plant cells can be derived from plants varying in age. The plant cells can be contacted with the biological device in a number of different ways. In one aspect, the device can be added to a medium containing the plant cells. In another aspect, the device can be injected into the plant cells via syringe. The amount of device and the duration of exposure to the device can vary as well. In one aspect, the concentration of the device is about 103, 104, 105, 106, 107, 108, or 109 cells/mL of water. In one aspect, when the host cell is a bacterium, the concentration of the device is 106. In another aspect, when the host cell is yeast, the concentration of the device is 109. Different volumes of the biological device can be used as well, ranging from 5 μL to 500 μL.

In certain aspects, any of the biological devices described above can be used in combination with a polysaccharide to enhance one or more physiological properties of the plant. In one aspect, the plant cells are first contacted with the biological device, then subsequently contacted with the polysaccharide. In another aspect, the plant cells are first contacted with the polysaccharide and subsequently contacted with the biological device. In a further aspect, the plant cells are simultaneously contacted with the polysaccharide and the biological device.

In one aspect, the polysaccharide includes chitosan, glucosamine (GlcN), N-acetylglucosamine (NAG), or any combination thereof. Chitosan is generally composed of glucosamine units and N-acetylglucosamine units and can be chemically or enzymatically extracted from chitin, which is a component of arthropod exoskeletons and fungal and microbial cell walls. In certain aspects, the chitosan can be acetylated to a specific degree of acetylation in order to enhance tissue growth during culturing as well as metabolite production. In one aspect, the chitosan is from 60% to about 100%, 70% to 90%, 75% to 85%, or about 80% acetylated. In one aspect, chitosan isolated from the shells of crab, shrimp, lobster, and/or krill is useful herein.

The molecular weight of the chitosan can vary, as well. For example, the chitosan comprises about 3, 4, 5, 6, 7, 8, 9, 10, 11, 12, 13, 14, 15, 16, 17, 18, 19, or 20 glucosamine units and/or N-acetylglucosamine units. In another aspect, the chitosan includes 5 to 7 glucosamine units and/or N-acetylglucosamine units. In one aspect, the chitosan is in a solution of water and acetic acid at less than 1% by weight, less than 0.75% by weight, less than 0.5% by weight, less than 0.25% by weight, or less than 0.1% by weight. In another aspect, the amount of chitosan that is applied to the plant cells is from 0.1% to 0.01% by weight, from 0.075% to 0.025% by weight, or is about 0.05% by weight. The polysaccharides used herein are generally natural polymers and thus present no environmental concerns. Additionally, the polysaccharide can be used in acceptably low concentrations.

The plant cells can be contacted with the polysaccharide using a number of techniques. In one aspect, the plant cells or reproductive organs (e.g., a plant embryo) can be cultured in agar and medium with a solution of the polysaccharide. In other aspects, the polysaccharide can be applied to a plant callus by techniques such as, for example, coating the callus or injecting the polysaccharide into the callus. In this aspect, the age of the callus can vary depending upon the type of plant. The amount of polysaccharide can vary depending upon, among other things, the selection and number of plant cells. The use of the polysaccharide in the methods described herein permits rapid tissue culturing at room temperature. Due to the ability of the polysaccharide to prevent microbial contamination, the tissue cultures can grow for extended periods of time ranging from days to several weeks. Moreover, tissue culturing with the polysaccharide can occur in the dark and/or light. As discussed above, the plant cells can be contacted with any of the biological devices described above. Thus, the use of the polysaccharides and biological devices described herein is a versatile way to culture and grow plant cells—and ultimately, plants of interest—with enhanced physiological properties.

In one aspect, a plant callus such as described above can be planted and allowed to grow and mature into a plant bearing fruit and leaves.

In a further aspect, provided herein is a plant grown by the process of contacting plant gamete cells, a plant reproductive organ, or a plant callus with the biological devices disclosed herein. Also provided herein is a method for producing such a plant. In one aspect, the method includes the steps of:
(a) contacting a plant callus with the biological device;
(b) culturing the plant callus; and
(c) growing a plant from the plant callus.

In some aspects, the plant callus is cultured with chitosan. In a further aspect, the chitosan is from 60%, 65%, 70%, 75%, 80%, 85%, 90%, 95%, or 100% acetylated and has 3, 4, 5, 6, 7, 8, 9, 10, 11, 12, 13, 14, 15, 16, 17, 18, 19, or 20 glucosamine units, N-acetylglucosamine units, or a combination thereof, where any value can be a lower and upper end-point of a range (e.g., 60% to 80% acetylation).

In another aspect, provided herein is a method for increasing the UV-resistance of a plant, the method involving growing a plant from plant cells that have been contacted with the biological devices disclosed herein. In one aspect, increased UV-resistance can be measured by growing plants from a treated and an untreated callus alongside one another and comparing UV-induced damage after a period of time. In a further aspect, an agricultural product harvested from a UV-resistant plant will also be more UV-resistant. Further in this aspect, for example, cotton from a cotton plant grown with the biological devices will be more UV-resistant than cotton grown from an untreated plant.

### Solar Cells

In other aspects, zinc oxide produced by the devices herein can be used to produce solar cells. Solar cells typically include a thin film of semiconductive inorganic material. Examples of such materials include titanium oxide and zinc oxide. In one aspect, zinc oxide produced by the biological devices described herein can be used as the semiconductive layer in a solar cell. For example, any of the devices described herein that include a gene that expresses zinc oxidase (e.g., device in FIG. 3) can be used to produce zinc oxide that can subsequently be used to produce solar cells.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. Numerous variations and combinations of reaction conditions, e.g., component concentrations, desired solvents, solvent mixtures, temperatures, pressures, and other reaction ranges and conditions can be used to optimize the product purity and yield obtained from the described process. Only reasonable and routine experimentation will be required to optimize such processes and conditions.

The DNA construct was composed of genetic components described herein and assembled in plasmid vectors (e.g., pYES2 and pBSK). Sequences of genes and/or proteins with desired properties were identified in GenBank; these included a DXP synthase gene, a beta-carotene hydroxylase gene, and a lycopene epsilon-cyclase gene. Other genetic parts were also obtained for inclusion in the DNA constructs including, for example, promoter genes (e.g., GAL1 promoter), reporter genes (e.g., yellow fluorescent reporter protein), and terminator sequences (e.g., CYC1 terminator). These genetic parts included restriction sites for ease of insertion into plasmid vectors.

The cloning of the DNA construct into the biological devices was performed as follows. Sequences of individual genes were amplified by polymerase chain reaction using primers that incorporated restriction sites at their 5′ ends to facilitate construction of the full sequence to be inserted into the plasmid. Genes were then ligated using standard protocols to form an insert. The plasmid was then digested with restriction enzymes according to directions and using reagents provided by the enzyme's supplier (Promega). The complete insert, containing restriction sites on each end, was then ligated into the plasmid. Successful construction of the insert and ligation of the insert into the plasmid were confirmed by gel electrophoresis.

PCR was used to enhance DNA concentration using a Mastercycler Personal 5332 ThermoCycler (Eppendorf North America) with specific sequence primers and the standard method for amplification (Sambrook, J., E. F. Fritsch, and T. Maniatis, 1989, Molecular Cloning: A Laboratory Manual, 2^{nd} ed., Vol. 1, Cold Spring Harbor Laboratory Press: Cold Spring Harbor, NY). Digestion and ligation were used to ensure assembly of DNA synthesized parts using restriction enzymes and reagents (PCR master mix of restriction enzymes: XhoI, KpnI, XbaI, EcoRI, BamHI, and HindIII, with alkaline phosphatase and quick ligation kit, all from Promega). DNA was quantified using a Nano Vue spectrophotometer (GE Life Sciences) and a standard UV/Visible spectrophotometer using the ratio of absorbances at 260 nm versus 280 nm. In order to verify final ligations, DNA was visualized and purified via electrophoresis using a Thermo EC-150 power supply.

The DNA construct was made with gene parts fundamental for expression of sequences such as, for example, ribosomal binding sites, native and constitutive promoters, reporter genes, and transcriptional terminators or stops. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. The DNA constructs in FIGS. 1-3 were assembled using the techniques above.

After the vector comprising the DNA construct has been produced, the resulting vector can be incorporated into the host cells using the methods known in the art (e.g., Gietz, R. D. and R. H. Schiestl, 2007, Nature Protocols, “Quick and easy yeast transformation using the LiAc/SS carrier DNA/PEG method,” Vol. 2, 35-37, doi: 10.1038/nprot.2007.14).

### Production of Anti-UV Extract

Yeast (Saccharomyces cerevisiae) transformed with the construct as provided in FIGS. 1A and 1B was fermented in yeast malt medium with 2% of raffinose, 1 mg/mL of glucosamine, and induced with 1% of galactose at 30° C. for 72 hours. After sonication for 150 minutes, the supernatant was sequentially filtered with 8 μm, 5 μm, 3 μm, 2 μm and 1.2 μm filters. The extract isolated from filtrations was used below in the testing below.

Procedure for Biological Test for Anti UV protection on Bacillus subtilis ATCC

A culture of Bacteria Bacillus subtilis (ATCC 82) was grown at 30° C. for one to two days. Aliquots were taken from this culture and subjected to different bacteria dilutions and the concentration was determined spectrophotometrically at the respective optical density, OD (i.e. 0.5, 1.0, 1.4, 2.0, with 1.4 as the preferred OD). Solutions were made from the above dilutions, and were mixed with different concentrations of the extract produced above at different proportions (i.e. 5:2 vol, extract: bacteria). These solutions were placed in Petri dishes with a total volume of 7 mL. These solutions were made in triplicates. Each solutions was placed in the UV incubator, and samples were taken at different times (i.e. 30 minutes, 1 hour, 2 hours).

Aliquots of 1 mL bacterial samples were taken from each replicate and fully mixed. Then, 500 μL were taken and placed on Nutrient agar by using standard streaking method, with 3 agar plate replicates were used at each time. The agar plates were incubated at 30° C. for 1-4 days. Bacterial colonies of Bacillus subtilis were viewed and counted at each time. The samples taken from the mixtures with no extract present showed essentially co colony growth; however, bacteria mixed with anti-UV extract showed considerable growth. The results indicate that the anti-UV extract can protect cells such as bacterial cells from exposure to UV light.

Procedure for Anti UV protection on Fibroblast Cells ATCC 2522-CRL

Skin fibroblast were used as a model for human skin and were maintained in culture media for propagation and renewal following ATCC recommendations. The propagation medium is based on ATCC-formulated Eagles's Minimum Essential Medium, Catalog No 30-2003-Fetal bovine serum was added to the medium to a final concentration of 10%. The medium was also renewed according to ATCC instructions. This medium is made Of 0.025% trypsin, 0.03% EDTA solution. Culture of fibroblast cells (ATCC 2522-CRL) was grown at 37° C. and 5% CO₂.

Extracts as produced above were applied to fibroblast culture with different ratios (volume) extract to fibroblast. This mixture was then exposed to UV-B radiation (302 nm) and UV-A (365 nm) for different times, and incubated at 37°° C. and 5% of CO2; each experiment was performed in triplicate.

Aliquots of fibroblast cells were harvested and subjected to microscopic analysis, carried out following standard procedure to count dead, living and apoptosis cells by staining the cells with trypan blue and viewing them under a compound microscope. Cells were counted at 20× magnification using several microscopic field views. Tables 9 and 10 provide the results of the experiment. The results indicate that the anti-UV extract can protect fibroblast cells from exposure to UV light.

TABLE 9
Percentage of alive, death, apoptosis fibroblast cells at different
times and total survival and protection of experiment.
Protective effect of Anti-UV Extract on Skin Cells, Against UV-A
at different times, proportion 5:4 (Extract:Skin cells culture)

| | | | SCC + Anti-UV | |
|---|---|---|---|---|
| | SCC + Water | | Extract | |
| | (Control) | | (Treatment) | |
| Type of cell | 30 minutes | 1 hour | 30 minutes | 1 hour |
| Live Cells (Percentage) | 0 | 0 | 20 | 14 |
| Death Cells (Percentage) | 67 | 87 | 0 | 29 |
| Apoptose Cells (Percentage) | 33 | 13 | 80 | 57 |
| Total Survival (Percentage) | 33 | 13 | 100 | 71 |
| Protection (Percentage) | — | — | 67 | 58 |

TABLE 10
Percentage of alive, death, apoptosis fibroblast cells at different
times and total survival and protection of experiment.
Protective effect of Anti UV Extract on Skin Cells, Against UV-B
at different times, proportion 5:4 (Extract:Skin cells culture)

| | SCC + Water | | SCC + Anti-UV | |
|---|---|---|---|---|
| | (Control) | | Extract | |
| Type of cell | 30 minutes | 1 hour | 30 minutes | 1 hour |
| Live Cells (Percentage) | 0 | 0 | 60 | 50 |
| Death Cells (Percentage) | 75 | 90 | 10 | 28 |
| Apoptose Cells (Percentage) | 25 | 10 | 30 | 22 |
| Total Survival (Percentage) | 25 | 10 | 90 | 72 |
| Protection (Percentage) | — | — | 65 | 62 |

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

What is claimed:

1. A topical composition comprising hexokinase, a heat shock protein, and alcohol dehydrogenase.

2. The topical composition of claim 1, wherein the heat shock protein is HSP70.

3. The topical composition of claim 1, wherein the composition further comprises transferrin.

4. The topical composition of claim 1, wherein the composition further comprises flavonol synthase.

5. The topical composition of claim 1, wherein the composition further comprises iron oxidase.

6. The topical composition of claim 1, wherein the composition further comprises zinc oxidase.

7. The topical composition of claim 1, wherein the composition further comprises flavonol synthase and iron oxidase.

8. The topical composition of claim 1, wherein the composition further comprises flavonol synthase and zinc oxidase.

9. The topical composition of claim 1, wherein the topical composition is a cosmetic.

10. The topical composition of claim 1, wherein the topical composition is a sunscreen.

11. The topical composition of claim 1, wherein the topical composition is a paste, lotion, cream, or aerosol.

12. The topical composition of claim 1, wherein the topical composition further comprises an emulsifier, a preservative, a sequestering agent, a fragrance, a thickener, an oil, a wax, or a film-forming polymer.

13. The topical composition of claim 1, wherein the topical composition further comprises one or more UV-protective compounds or UV-blocking agents.

14. The topical composition of claim 1, wherein hexokinase, a heat shock protein, and alcohol dehydrogenase in the topical composition is from 0.01 g per mL of the composition to 1 g per mL of the composition.

15. A plant or agricultural product coated with the topical composition of claim 1.

16. A method of reducing or preventing exposure of a subject to UV radiation comprising applying to the subject the topical composition of claim 1.

17. A paint, ink, dye, or stain comprising hexokinase, a heat shock protein, and alcohol dehydrogenase.

18. An article coated with a mixture comprising hexokinase, a heat shock protein, and alcohol dehydrogenase, the comprising hexokinase, a heat shock protein, and alcohol dehydrogenase is dispersed throughout the article, or a combination thereof.

19. The article of claim 17, wherein the article is made of glass, fiberglass, plastic, metal, wood, fabric, foam, rubber, latex, silicone, or any combination thereof.


SEQUENCE LISTING (12 sequences)

SEQ ID NO: 1 - DNA, 16377 bp, synthetic construct
  source 1..16377: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctagggtttaattaagtggcgcgccatggttcatttaggtccaaagaaaccacaggctagaaagggttccatggctgatgtgcccaaggaattgatggatgaaattcatcagttggaagatatgtttacagttgacagcgagaccttgagaaaggttgttaagcactttatcgacgaattgaataaaggtttgacaaagaagggaggtaacattccaatgattcccggttgggtcatggaattcccaacaggtaaagaatctggtaactatttggccattgatttgggtggtactaacttaagagtcgtgttggtcaagttgagcggtaaccatacctttgacaccactcaatccaagtataaactaccacatgacatgagaaccactaagcaccaagaggagttatggtcctttattgccgactctttgaaggactttatggtcgagcaagaattgctaaacaccaaggacaccttaccattaggtttcaccttctcgtacccagcttcccaaaacaagattaacgaaggtattttgcaaagatggaccaagggtttcgatattccaaatgtcgaaggccacgatgtcgtcccattgctacaaaacgaaatttccaagagagagttgcctattgaaattgtagcattgattaatgatactgttggtactttaattgcctcatactacactgacccagagactaagatgggtgtgattttcggtactggtgtcaacggtgctttctatgatgttgtttccgatatcgaaaagttggagggcaaattagcagacgatattccaagtaactctccaatggctatcaattgtgaatatggttccttcgataatgaacatttggtcttgccaagaaccaagtacgatgttgctgtcgacgaacaatctccaagacctggtcaacaagcttttgaaaagatgacctccggttactacttgggtgaattgttgcgtctagtgttacttgaattaaacgagaagggcttgatgttgaaggatcaagatctaagcaagttgaaacaaccatacatcatggatacctcctacccagcaagaatcgaggatgatccatttgaaaacttggaagatactgatgacatcttccaaaaggactttggtgtcaagaccactctgccagaacgtaagttgattagaagactttgtgaattgatcggtaccagagctgctagattagctgtttgtggtattgccgctatttgccaaaagagaggttacaagactggtcacattgccgctgacggttctgtctataacaaatacccaggtttcaaggaagccgccgctaagggtttgagagatatctatggatggactggtgacgcaagcaaagatccaattacgattgttccagctgaggatggttcaggtgcaggtgctgctgttattgctgcattgtccgaaaaaagaattgccgaaggtaagtctcttggtatcattggcgcttaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatcgtacgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcatgctagtagttaggtgccctggccgttcccagcattggtggccgcggccgtggccggaccggcgcccggggtggcgcccagcaccgactgcaggtcaggcttcatcgcctgcagctgcgcgccccagtagggccagtcgtgggtgccgttggcgtcgaagttccacaccgcgttgtggccgccggcgccgttgtaggcgtcctggaacttcaggttggacgtccgcacgaagccctcgaggaacttggcgggcaggttgtcgccaccgaggtcggacggcttgccgttgccgcagtacacccagatccgggtgttgttcgcgaccagcttgccgacctgcagcgacgggtcgttgcgggcccaggccgggtcctccttcggaccccacatgtcggcggccttgtagccaccggcgtcacccatggccagcccgatcagcgacgggcccatgccctgcgacgagtccagcagcgccgacagcgagccggcgtagacgaactggtcggggtggtaggcggccaggatcagcgccgaggagccggccatcgacaggccgacgacaccgctgccggtcggcttgacctgcttctgcgccgacaggtactgcggcagctcgctggtcaggaaggtctcccacttgtaggtggtgcagccggccttgccgcaggcgggcttgtaccagtcggagtagaagctggactggccgccgaccggcatggcgaccgagatgcccgactggttgtaccactcgaacgccggggtgttgatgtcccagccgttgaagtcgtcttgcgcgcgcatcccgtcgagcaggtacaacgcgggcgagttggccccaccgctttggaactggaccttgatgtcccgtcccatggcggcggagggaacctgcaggtactccaccggcagaccggggcgcgagaaggccccggcggtcgccgagcccccgacggcgccaatcaggcccgagagcagcgccgcaccagcggcccccaccacgagccggcgcggcatccccgccacggcgccgcgcaatctgtcgacatgcgtctggccgttggtgccctgctggtgtgcgcggtgctgggcctgtgcctggccgtcccagataaaaccgttcgctggtgcgcagtcagtgagcatgaagcgacgaagtgccaatctttccgcgatcacatgaaaagcgtaattccgagcgatggtccgagtgtagcttgtgttaagaaagcaagctatctggactgtatccgcgcaattgcagcgaacgaagctgatgcagttaccctggacgcaggtctggtttacgacgcgtacctggctcctaacaatctgaaaccggttgtagcggagttctatggtagtaaagaagacccgcaaactttctattatgcagtggccgtggtaaagaaggactctggttttcagatgaaccagctgcgtgggaagaaaagttgtcatacgggcctggggcgttctgcgggttggaacattccaattgggctgctgtattgcgatctgccggaaccacgcaagccgctggagaaagctgtagcgaacttcttcagtggttcttgtgcgccttgcgccgatggtactgattttccgcagctgtgtcagctgtgtccgggctgcggttgttctaccctgaatcagtactttggctatagtggggcgtttaaatgcctgaaagatggggcgggtgacgtggcgttcgtcaaacattctacgattttcgaaaacctggcgaacaaagcagatcgtgaccaatatgaactgctgtgtctggacaacactcgcaagccagtcgatgaatataaagattgtcatctggcacaagtgcctagtcatactgtggtcgcgcgtagcattggtggtaaggaggacctgatttgggaactgctgaaccaagctcaggagcatttcggcaaagataaaagcaaagaatttcagctgttttctagcccgcacggcaaagacctgctgtttaaagacagcgcccacggctttctgaaagtgcctccacgcatggatgccaaaatgtatctgggttatgaatatgttacggcaattcgcaatctgcgtgaaggcacgtgcccggaagctccgactgacgagtgcaaaccagtaaagtggtgtgccctgtctcatcatgagcgcctgaaatgtgatgaatggagtgtgaactctgttggcaaaattgagtgcgttagtgctgaaaccaccgaggactgtatcgcaaagatcatgaacggcgaagcagatgctatgtctctggatggcggttttgtgtatatcgcaggtaaatgcggcctggtcccagttctggctgaaaattataacaaaagtgataactgtgaggatactccaggggcgggctattttgcggtcgctgtcgtcaagaaatctgcgagcgatctgacatgggataacctgaaagggaagaaatcttgccataccgcggttggccgcaccgctgggtggaacatcccgatgggcctgctgtataacaaaatcaatcattgccgttttgacgagttcttcagtgaggggtgtgcgcctggtagtaagaaagatagcagcctgtgcaaactgtgcatgggcagcggcctgaatctgtgcgaacctaacaataaagagggttactacggctacaccggcgcgtttcgctgcctggttgagaaaggtgatgttgcgtttgtaaagcaccaaacagtaccgcagaatacgggtgggaagaatccggacccgtgggccaagaatctgaatgaaaaggattacgaactgctgtgcctggatgggacccgcaagccggttgaagaatacgcgaattgtcacctggcccgcgccccgaatcacgccgtggtgacgcgcaaagataaagaggcctgcgtccacaaaatcctgcgtcagcagcagcacctgttcggcagcaatgtgacagattgtagcggtaatttctgtctgttccgtagcgaaaccaaggacctgctgttccgtgacgacaccgtgtgtctggccaaactgcacgaccgtaatacctacgagaaatacctgggcgaggagtacgtgaaagccgtgggcaatctgcgtaagtgtagcacaagcagcctgctggaagcctgcacatttcgtcgtccgtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 2 - DNA, 16292 bp, synthetic construct
  source 1..16292: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggatggaggtcgaaagagtccaagacatttcatcttcttctctactaacagaagcaatcccgttggagttcatcagatcagagaaagaacaaccagcgatcacaacattccgaggtccaacgccggcgattcccgtcgtcgatctaagcgatcccgacgaagaaagcgtgaggcgcgcggtggtgaaagcgagtgaagaatgggggctattccaagtggttaaccacgggattccgacggagctgatacgacgtttacaagacgtcggaagaaaattcttcgagcttccttcgtcggagaaagaatccgtcgctaaaccggaagattcgaaagacattgaaggatacggaacaaagcttcagaaagatccagaaggtaaaaaagcttgggtcgatcatctcttccatcgaatctggccaccgtcatgcgtcaattacagattctggcctaagaatccacctgaatacagggaggtgaatgaagagtatgcagtgcatgtgaagaagctatcggagacgttattagggattctctcggatggattagggttaaagcgtgatgcgttgaaagaaggtctcggcggagagatggcggagtatatgatgaagattaactattatccgccgtgtcctcggccggatttagctttaggtgtaccggctcatacagatctcagtggaatcactcttcttgttcctaacgaagttcctggacttcaagttttcaaagatgatcactggttcgatgcagagtatattccctccgccgtcattgttcacatcggcgatcagattctgaggttgagtaatgggaggtataaaaatgtgttgcataggacgacggtggataaagagaagacgaggatgtcgtggccggttttcttggagcctccccgtgaaaagattgttggacctttaccggaactaaccggagatgataatcctccaaagtttaaaccgtttgctttcaaggattacagttaccgcaagctcaataaacttcctctggattgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccatggttcatttaggtccaaagaaaccacaggctagaaagggttccatggctgatgtgcccaaggaattgatggatgaaattcatcagttggaagatatgtttacagttgacagcgagaccttgagaaaggttgttaagcactttatcgacgaattgaataaaggtttgacaaagaagggaggtaacattccaatgattcccggttgggtcatggaattcccaacaggtaaagaatctggtaactatttggccattgatttgggtggtactaacttaagagtcgtgttggtcaagttgagcggtaaccatacctttgacaccactcaatccaagtataaactaccacatgacatgagaaccactaagcaccaagaggagttatggtcctttattgccgactctttgaaggactttatggtcgagcaagaattgctaaacaccaaggacaccttaccattaggtttcaccttctcgtacccagcttcccaaaacaagattaacgaaggtattttgcaaagatggaccaagggtttcgatattccaaatgtcgaaggccacgatgtcgtcccattgctacaaaacgaaatttccaagagagagttgcctattgaaattgtagcattgattaatgatactgttggtactttaattgcctcatactacactgacccagagactaagatgggtgtgattttcggtactggtgtcaacggtgctttctatgatgttgtttccgatatcgaaaagttggagggcaaattagcagacgatattccaagtaactctccaatggctatcaattgtgaatatggttccttcgataatgaacatttggtcttgccaagaaccaagtacgatgttgctgtcgacgaacaatctccaagacctggtcaacaagcttttgaaaagatgacctccggttactacttgggtgaattgttgcgtctagtgttacttgaattaaacgagaagggcttgatgttgaaggatcaagatctaagcaagttgaaacaaccatacatcatggatacctcctacccagcaagaatcgaggatgatccatttgaaaacttggaagatactgatgacatcttccaaaaggactttggtgtcaagaccactctgccagaacgtaagttgattagaagactttgtgaattgatcggtaccagagctgctagattagctgtttgtggtattgccgctatttgccaaaagagaggttacaagactggtcacattgccgctgacggttctgtctataacaaatacccaggtttcaaggaagccgccgctaagggtttgagagatatctatggatggactggtgacgcaagcaaagatccaattacgattgttccagctgaggatggttcaggtgcaggtgctgctgttattgctgcattgtccgaaaaaagaattgccgaaggtaagtctcttggtatcattggcgcttaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatcgtacgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcatgctagtagttaggtgccctggccgttcccagcattggtggccgcggccgtggccggaccggcgcccggggtggcgcccagcaccgactgcaggtcaggcttcatcgcctgcagctgcgcgccccagtagggccagtcgtgggtgccgttggcgtcgaagttccacaccgcgttgtggccgccggcgccgttgtaggcgtcctggaacttcaggttggacgtccgcacgaagccctcgaggaacttggcgggcaggttgtcgccaccgaggtcggacggcttgccgttgccgcagtacacccagatccgggtgttgttcgcgaccagcttgccgacctgcagcgacgggtcgttgcgggcccaggccgggtcctccttcggaccccacatgtcggcggccttgtagccaccggcgtcacccatggccagcccgatcagcgacgggcccatgccctgcgacgagtccagcagcgccgacagcgagccggcgtagacgaactggtcggggtggtaggcggccaggatcagcgccgaggagccggccatcgacaggccgacgacaccgctgccggtcggcttgacctgcttctgcgccgacaggtactgcggcagctcgctggtcaggaaggtctcccacttgtaggtggtgcagccggccttgccgcaggcgggcttgtaccagtcggagtagaagctggactggccgccgaccggcatggcgaccgagatgcccgactggttgtaccactcgaacgccggggtgttgatgtcccagccgttgaagtcgtcttgcgcgcgcatcccgtcgagcaggtacaacgcgggcgagttggccccaccgctttggaactggaccttgatgtcccgtcccatggcggcggagggaacctgcaggtactccaccggcagaccggggcgcgagaaggccccggcggtcgccgagcccccgacggcgccaatcaggcccgagagcagcgccgcaccagcggcccccaccacgagccggcgcggcatccccgccacggcgccgcgcaatctgtcgacatgtcaaaggaacagaacggtacagagaagccacagaatttgagtagaagggacgtcttgaagggtattgcaatcacagcaggtgttgttgctgctggcgctgtagtgggagttaaccctattggtgctgctcatgctgctggtaagtgcccaggtatcactcctaaggcttcattacaataccaaccacaccctaaaggtaaagagcaatgctctgcctgtgcaaacttcatcgcaccacattgttgtaaagtggttgctggttctgttgttccagaaggatattgtatggccttcatcttgaagcctgcataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 3 - DNA, 16760 bp, synthetic construct
  source 1..16760: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggatgacagatcgttgtccaggtagggatgctccacacttagcagtcattggagcaggtccagctggcttagcagcagcattagctgctgctgctagaggtgttcgtgtaaccttgttggatgctgaaccagaagcaggaggccaattctatagacagccagcagcagctttacgtgctagaaggccacaagcattacaccatcagtggcgtacctttgccagattgagacacggattagccaggcacattgcagcaggtagagttagacatgctagagaacaccatgtttggtttgctgagagagctcctgatggtggattcaccgttcatgctttgactggtccaggtagaggagatccagcagaagtgagagcagatgcagtcttgttggcaactggtggtcacgagactgtgttgccattcccaggttggaccttgccaggtgttgtcacagctggaggtgcccaagccatgttgaaggcaggtttagttacatctggcaacaccgcagtcgtagctggtactggtccattgttgttgccagtagctacaggtttagctgctgctggtgttgacgtaagagcattagtcgaaagtgctgatcctggtgccttaccaagacaggcacgtgctttggcagctcaacctggcaagttggctgaaggtgctttgtatgctggtcaattgttgaggcacagagtgcgtgtcttgactagacacactgtcgttgaagcacatggtacagagaggttggaagcagttactgttgcagccttggatgcaggtggacgtactagacctggcactgctagaagaatagcatgtgcaactttagctgtgggtcatggtatgttgccacatacagacttggcagacgccttaggctgccgtttagcaggtccagcagttcatgcagatgatgaacaaagaactgatgttcctggtgtgtgggcagcaggagagtgtactggcgtaggtggtgcagctttgtctttggctgagggtcatatcgctggcagaagtgcagcagccagattgttaggagcacctccaggtcccgacgcatggccagaggcagctagaacaagagcaaggttgagagctttctccgctgtattggatgctgtttacactcctcctcctggttggggtgagagagtcaccgacgcaaccgttgtatgcaggtgtgaagaagttacagcaggtgcaatccgtgcttctgtgagggaattgggagctggtgacgtacgtactgtaaagttgttgactagagctggcatgggatggtgtcagggaagaatgtgtgctcctgctgtcgctggattggcaggttgtgctttcactcctagtcgtagaccattcgctaggccagtgcctttgggagtgttggccagagctggtgaagatgcaggtggcgatggaggcagagctgaggatcaaggtgaaggagatggacgtgctgctggagcaggaggttgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccatggttcatttaggtccaaagaaaccacaggctagaaagggttccatggctgatgtgcccaaggaattgatggatgaaattcatcagttggaagatatgtttacagttgacagcgagaccttgagaaaggttgttaagcactttatcgacgaattgaataaaggtttgacaaagaagggaggtaacattccaatgattcccggttgggtcatggaattcccaacaggtaaagaatctggtaactatttggccattgatttgggtggtactaacttaagagtcgtgttggtcaagttgagcggtaaccatacctttgacaccactcaatccaagtataaactaccacatgacatgagaaccactaagcaccaagaggagttatggtcctttattgccgactctttgaaggactttatggtcgagcaagaattgctaaacaccaaggacaccttaccattaggtttcaccttctcgtacccagcttcccaaaacaagattaacgaaggtattttgcaaagatggaccaagggtttcgatattccaaatgtcgaaggccacgatgtcgtcccattgctacaaaacgaaatttccaagagagagttgcctattgaaattgtagcattgattaatgatactgttggtactttaattgcctcatactacactgacccagagactaagatgggtgtgattttcggtactggtgtcaacggtgctttctatgatgttgtttccgatatcgaaaagttggagggcaaattagcagacgatattccaagtaactctccaatggctatcaattgtgaatatggttccttcgataatgaacatttggtcttgccaagaaccaagtacgatgttgctgtcgacgaacaatctccaagacctggtcaacaagcttttgaaaagatgacctccggttactacttgggtgaattgttgcgtctagtgttacttgaattaaacgagaagggcttgatgttgaaggatcaagatctaagcaagttgaaacaaccatacatcatggatacctcctacccagcaagaatcgaggatgatccatttgaaaacttggaagatactgatgacatcttccaaaaggactttggtgtcaagaccactctgccagaacgtaagttgattagaagactttgtgaattgatcggtaccagagctgctagattagctgtttgtggtattgccgctatttgccaaaagagaggttacaagactggtcacattgccgctgacggttctgtctataacaaatacccaggtttcaaggaagccgccgctaagggtttgagagatatctatggatggactggtgacgcaagcaaagatccaattacgattgttccagctgaggatggttcaggtgcaggtgctgctgttattgctgcattgtccgaaaaaagaattgccgaaggtaagtctcttggtatcattggcgcttaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatcgtacgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcatgctagtagttaggtgccctggccgttcccagcattggtggccgcggccgtggccggaccggcgcccggggtggcgcccagcaccgactgcaggtcaggcttcatcgcctgcagctgcgcgccccagtagggccagtcgtgggtgccgttggcgtcgaagttccacaccgcgttgtggccgccggcgccgttgtaggcgtcctggaacttcaggttggacgtccgcacgaagccctcgaggaacttggcgggcaggttgtcgccaccgaggtcggacggcttgccgttgccgcagtacacccagatccgggtgttgttcgcgaccagcttgccgacctgcagcgacgggtcgttgcgggcccaggccgggtcctccttcggaccccacatgtcggcggccttgtagccaccggcgtcacccatggccagcccgatcagcgacgggcccatgccctgcgacgagtccagcagcgccgacagcgagccggcgtagacgaactggtcggggtggtaggcggccaggatcagcgccgaggagccggccatcgacaggccgacgacaccgctgccggtcggcttgacctgcttctgcgccgacaggtactgcggcagctcgctggtcaggaaggtctcccacttgtaggtggtgcagccggccttgccgcaggcgggcttgtaccagtcggagtagaagctggactggccgccgaccggcatggcgaccgagatgcccgactggttgtaccactcgaacgccggggtgttgatgtcccagccgttgaagtcgtcttgcgcgcgcatcccgtcgagcaggtacaacgcgggcgagttggccccaccgctttggaactggaccttgatgtcccgtcccatggcggcggagggaacctgcaggtactccaccggcagaccggggcgcgagaaggccccggcggtcgccgagcccccgacggcgccaatcaggcccgagagcagcgccgcaccagcggcccccaccacgagccggcgcggcatccccgccacggcgccgcgcaatctgtcgacatgtcaaaggaacagaacggtacagagaagccacagaatttgagtagaagggacgtcttgaagggtattgcaatcacagcaggtgttgttgctgctggcgctgtagtgggagttaaccctattggtgctgctcatgctgctggtaagtgcccaggtatcactcctaaggcttcattacaataccaaccacaccctaaaggtaaagagcaatgctctgcctgtgcaaacttcatcgcaccacattgttgtaaagtggttgctggttctgttgttccagaaggatattgtatggccttcatcttgaagcctgcataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 4 - DNA, 1470 bp, Saccharomyces cerevisiae
  source 1..1470: mol_type = other DNA
gagctcatggttcatttaggtccaaagaaaccacaggctagaaagggttccatggctgatgtgcccaaggaattgatggatgaaattcatcagttggaagatatgtttacagttgacagcgagaccttgagaaaggttgttaagcactttatcgacgaattgaataaaggtttgacaaagaagggaggtaacattccaatgattcccggttgggtcatggaattcccaacaggtaaagaatctggtaactatttggccattgatttgggtggtactaacttaagagtcgtgttggtcaagttgagcggtaaccatacctttgacaccactcaatccaagtataaactaccacatgacatgagaaccactaagcaccaagaggagttatggtcctttattgccgactctttgaaggactttatggtcgagcaagaattgctaaacaccaaggacaccttaccattaggtttcaccttctcgtacccagcttcccaaaacaagattaacgaaggtattttgcaaagatggaccaagggtttcgatattccaaatgtcgaaggccacgatgtcgtcccattgctacaaaacgaaatttccaagagagagttgcctattgaaattgtagcgttgattaatgatactgttggtactttaattgcctcatactacactgacccagagactaagatgggtgtgattttcggtactggtgtcaacggtgctttctatgatgttgtttccgatatcgaaaagttggagggcaaattagcagacgatattccaagtaactctccaatggctatcaattgtgaatatggttccttcgataatgaacatttggtcttgccaagaaccaagtacgatgttgctgtcgacgaacaatctccaagacctggtcaacaagcttttgaaaagatgacctccggttactacttgggtgaattgttgcgtctagtgttacttgaattaaacgagaagggcttgatgttgaaggatcaagatctaagcaagttgaaacaaccatacatcatggatacctcctacccagcaagaatcgaggatgatccatttgttttcttggaagatactgatgacatcttccaaaaggactttggtgtcaagaccactctgccagaacgtaagttgattagaagactttgtgaattgactggtaccagagctgctagattagctgtttgtggtattgccgctatttgccaaaagagaggttacaagactggtcacattgccgctgacggttctgtctataacaaatacccaggtttcaaggaagccgccgctaagggtttgagagatatctatggatggactggtgacgcaagcaaagatccaattacgattgttccagctgaggatggttcaggtgcaggtgctgctgttattgctgcattgtccgaaaaaagaattgccgaaggtaagtctcttggtatcattggcgcttaactcgag

SEQ ID NO: 5 - DNA, 1854 bp, Saccharomyces cerevisiae
  source 1..1854: mol_type = other DNA
ggtaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaactcgag

SEQ ID NO: 6 - DNA, 1059 bp, Saccharomyces cerevisiae
  source 1..1059: mol_type = other DNA
ggtaccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtcaggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataactcgag

SEQ ID NO: 7 - DNA, 1097 bp, Mycobacterium avium
  source 1..1097: mol_type = other DNA
aagcttctgcagcggccgctactagtagttaggtgccctggccgttcccagcattggtggccgcggccgtggccggaccggcgcccggggtggcgcccagcaccgactgcaggtcaggcttcatcgcctgcagctgcgcgccccagtagggccagtcgtgggtgccgttggcgtcgaagttccacaccgcgttgtggccgccggcgccgttgtaggcgtcctggaacttcaggttggacgtccgcacgaagccctcgaggaacttggcgggcaggttgtcgccaccgaggtcggacggcttgccgttgccgcagtacacccagatccgggtgttgttcgcgaccagcttgccgacctgcagcgacgggtcgttgcgggcccaggccgggtcctccttcggaccccacatgtcggcggccttgtagccaccggcgtcacccatggccagcccgatcagcgacgggcccatgccctgcgacgagtccagcagcgccgacagcgagccggcgtagacgaactggtcggggtggtaggcggccaggatcagcgccgaggagccggccatcgacaggccgacgacaccgctgccggtcggcttgacctgcttctgcgccgacaggtactgcggcagctcgctggtcaggaaggtctcccacttgtaggtggtgcagccggccttgccgcaggcgggcttgtaccagtcggagtagaagctggactggccgccgaccggcatggcgaccgagatgcccgactggttgtaccactcgaacgccggggtgttgatgtcccagccgttgaagtcgtcttgcgcgcgcatcccgtcgagcaggtacaacgcgggcgagttggccccaccgctttggaactggaccttgatgtcccgtcccatggcggcggagggaacctgcaggtactccaccggcagaccggggcgcgagaaggccccggcggtcgccgagcccccgacggcgccaatcaggcccgagagcagcgccgcaccagcggcccccaccacgagccggcgcggcatccccgccacggcgccgcgcaatctgtcgacaagcgtcatctagaagcggccgcgaattcggatc

SEQ ID NO: 8 - DNA, 2130 bp, Homo sapiens
  source 1..2130: mol_type = other DNA
agatctaaagaggagaaaatgcgtctggccgttggtgccctgctggtgtgcgcggtgctgggcctgtgcctggccgtcccagataaaaccgttcgctggtgcgcagtcagtgagcatgaagcgacgaagtgccaatctttccgcgatcacatgaaaagcgtaattccgagcgatggtccgagtgtagcttgtgttaagaaagcaagctatctggactgtatccgcgcaattgcagcgaacgaagctgatgcagttaccctggacgcaggtctggtttacgacgcgtacctggctcctaacaatctgaaaccggttgtagcggagttctatggtagtaaagaagacccgcaaactttctattatgcagtggccgtggtaaagaaggactctggttttcagatgaaccagctgcgtgggaagaaaagttgtcatacgggcctggggcgttctgcgggttggaacattccaattgggctgctgtattgcgatctgccggaaccacgcaagccgctggagaaagctgtagcgaacttcttcagtggttcttgtgcgccttgcgccgatggtactgattttccgcagctgtgtcagctgtgtccgggctgcggttgttctaccctgaatcagtactttggctatagtggggcgtttaaatgcctgaaagatggggcgggtgacgtggcgttcgtcaaacattctacgattttcgaaaacctggcgaacaaagcagatcgtgaccaatatgaactgctgtgtctggacaacactcgcaagccagtcgatgaatataaagattgtcatctggcacaagtgcctagtcatactgtggtcgcgcgtagcattggtggtaaggaggacctgatttgggaactgctgaaccaagctcaggagcatttcggcaaagataaaagcaaagaatttcagctgttttctagcccgcacggcaaagacctgctgtttaaagacagcgcccacggctttctgaaagtgcctccacgcatggatgccaaaatgtatctgggttatgaatatgttacggcaattcgcaatctgcgtgaaggcacgtgcccggaagctccgactgacgagtgcaaaccagtaaagtggtgtgccctgtctcatcatgagcgcctgaaatgtgatgaatggagtgtgaactctgttggcaaaattgagtgcgttagtgctgaaaccaccgaggactgtatcgcaaagatcatgaacggcgaagcagatgctatgtctctggatggcggttttgtgtatatcgcaggtaaatgcggcctggtcccagttctggctgaaaattataacaaaagtgataactgtgaggatactccaggggcgggctattttgcggtcgctgtcgtcaagaaatctgcgagcgatctgacatgggataacctgaaagggaagaaatcttgccataccgcggttggccgcaccgctgggtggaacatcccgatgggcctgctgtataacaaaatcaatcattgccgttttgacgagttcttcagtgaggggtgtgcgcctggtagtaagaaagatagcagcctgtgcaaactgtgcatgggcagcggcctgaatctgtgcgaacctaacaataaagagggttactacggctacaccggcgcgtttcgctgcctggttgagaaaggtgatgttgcgtttgtaaagcaccaaacagtaccgcagaatacgggtgggaagaatccggacccgtgggccaagaatctgaatgaaaaggattacgaactgctgtgcctggatgggacccgcaagccggttgaagaatacgcgaattgtcacctggcccgcgccccgaatcacgccgtggtgacgcgcaaagataaagaggcctgcgtccacaaaatcctgcgtcagcagcagcacctgttcggcagcaatgtgacagattgtagcggtaatttctgtctgttccgtagcgaaaccaaggacctgctgttccgtgacgacaccgtgtgtctggccaaactgcacgaccgtaatacctacgagaaatacctgggcgaggagtacgtgaaagccgtgggcaatctgcgtaagtgtagcacaagcagcctgctggaagcctgcacatttcgtcgtccgtaaggatcctcagaattc

SEQ ID NO: 9 - DNA, 1025 bp, Fragaria X ananassa
  source 1..1025: mol_type = other DNA
cctaggatggaggtcgaaagagtccaagacatttcatcttcttctctactaacagaagcaatcccgttggagttcatcagatcagagaaagaacaaccagcgatcacaacattccgaggtccaacgccggcgattcccgtcgtcgatctaagcgatcccgacgaagaaagcgtgaggcgcgcggtggtgaaagcgagtgaagaatgggggctattccaagtggttaaccacgggattccgacggagctgatacgacgtttacaagacgtcggaagaaaattcttcgagcttccttcgtcggagaaagaatccgtcgctaaaccggaagattcgaaagacattgaaggatacggaacaaagcttcagaaagatccagaaggtaaaaaagcttgggtcgatcatctcttccatcgaatctggccaccgtcatgcgtcaattacagattctggcctaagaatccacctgaatacagggaggtgaatgaagagtatgcagtgcatgtgaagaagctatcggagacgttattagggattctctcggatggattagggttaaagcgtgatgcgttgaaagaaggtctcggcggagagatggcggagtatatgatgaagattaactattatccgccgtgtcctcggccggatttagctttaggtgtaccggctcatacagatctcagtggaatcactcttcttgttcctaacgaagttcctggacttcaagttttcaaagatgatcactggttcgatgcagagtatattccctccgccgtcattgttcacatcggcgatcagattctgaggttgagtaatgggaggtataaaaatgtgttgcataggacgacggtggataaagagaagacgaggatgtcgtggccggttttcttggagcctccccgtgaaaagattgttggacctttaccggaactaaccggagatgataatcctccaaagtttaaaccgtttgctttcaaggattacagttaccgcaagctcaataaacttcctctggattgattaattaa

SEQ ID NO: 10 - DNA, 327 bp, Acidithiobacillus ferrivorans
  source 1..327: mol_type = other DNA
gtcgacatgtcaaaggaacagaacggtacagagaagccacagaatttgagtagaagggacgtcttgaagggtattgcaatcacagcaggtgttgttgctgctggcgctgtagtgggagttaaccctattggtgctgctcatgctgctggtaagtgcccaggtatcactcctaaggcttcattacaataccaaccacaccctaaaggtaaagagcaatgctctgcctgtgcaaacttcatcgcaccacattgttgtaaagtggttgctggttctgttgttccagaaggatattgtatggccttcatcttgaagcctgcataatctaga

SEQ ID NO: 11 - DNA, 1493 bp, Streptomyces zinciresistens
  source 1..1493: mol_type = other DNA
cctaggatgacagatcgttgtccaggtagggatgctccacacttagcagtcattggagcaggtccagctggcttagcagcagcattagctgctgctgctagaggtgttcgtgtaaccttgttggatgctgaaccagaagcaggaggccaattctatagacagccagcagcagctttacgtgctagaaggccacaagcattacaccatcagtggcgtacctttgccagattgagacacggattagccaggcacattgcagcaggtagagttagacatgctagagaacaccatgtttggtttgctgagagagctcctgatggtggattcaccgttcatgctttgactggtccaggtagaggagatccagcagaagtgagagcagatgcagtcttgttggcaactggtggtcacgagactgtgttgccattcccaggttggaccttgccaggtgttgtcacagctggaggtgcccaagccatgttgaaggcaggtttagttacatctggcaacaccgcagtcgtagctggtactggtccattgttgttgccagtagctacaggtttagctgctgctggtgttgacgtaagagcattagtcgaaagtgctgatcctggtgccttaccaagacaggcacgtgctttggcagctcaacctggcaagttggctgaaggtgctttgtatgctggtcaattgttgaggcacagagtgcgtgtcttgactagacacactgtcgttgaagcacatggtacagagaggttggaagcagttactgttgcagccttggatgcaggtggacgtactagacctggcactgctagaagaatagcatgtgcaactttagctgtgggtcatggtatgttgccacatacagacttggcagacgccttaggctgccgtttagcaggtccagcagttcatgcagatgatgaacaaagaactgatgttcctggtgtgtgggcagcaggagagtgtactggcgtaggtggtgcagctttgtctttggctgagggtcatatcgctggcagaagtgcagcagccagattgttaggagcacctccaggtcccgacgcatggccagaggcagctagaacaagagcaaggttgagagctttctccgctgtattggatgctgtttacactcctcctcctggttggggtgagagagtcaccgacgcaaccgttgtatgcaggtgtgaagaagttacagcaggtgcaatccgtgcttctgtgagggaattgggagctggtgacgtacgtactgtaaagttgttgactagagctggcatgggatggtgtcagggaagaatgtgtgctcctgctgtcgctggattggcaggttgtgctttcactcctagtcgtagaccattcgctaggccagtgcctttgggagtgttggccagagctggtgaagatgcaggtggcgatggaggcagagctgaggatcaaggtgaaggagatggacgtgctgctggagcaggaggttgattaattaa

SEQ ID NO: 12 - DNA, 890 bp, synthetic construct
  source 1..890: mol_type = other DNA
ggattcaaagaggagaaatactagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatactagagccaggcatcaaataaaacgaaaggctcagtcgaaagactgggcctttcgttttatctgttgtttgtcggtgaacgctctctactagagtcacactggctcaccttcgggtgggcctttctgcgtttataaagctt